(19) United States
(12) Patent Application Publication
(10) Pub. No.: US 2010/0114292 A1
(43) Pub. Date: May 6, 2010

(54) Vessel Extender

(75) Inventors: Larry C. Heaton, II, Pleasanton, CA (US); Alex Keller, Manhasset, NY (US)

Correspondence Address: AKA CHAN LLP, 900 LAFAYETTE STREET, SUITE 710, SANTA CLARA, CA, 95050, US

(73) Assignee: VIOPTIX, INC., Fremont, CA (US)

(21) Appl. No.: 12/263,053

(22) Filed: Oct. 31, 2008

(51) Int. Cl. A61F 2/44

(52) U.S. Cl. 623/1.15; 623/1.32; 623/1.36; 623/1.51

(57) ABSTRACT

An anastomotic device includes a tube and anastomotic coupler rings having vessel openings attached to each end of the tube. In an embodiment, the diameters of the vessel openings are the same.

### BACKGROUND OF THE INVENTION

The present invention relates to the field of medical devices, and more specifically, to an artificial or natural body vessel with coupler rings.

Medical devices are among the marvels of modern medicine. Doctors and people use medical devices to help treat patients who suffer from injuries, diseases, and the consequences of old age. Some revolutionary medical devices include the balloon catheter, oximeter, stent, and shunt. Over the years, these have improved the lives of many millions of people—allowing them to live better, longer, and more fulfilling lives.

Medical devices continue to evolve and improve. Today's medical devices are more durable, dependable, and easier to use than those introduced just a few years ago.

Despite the widespread success of current medical devices, there is a need for new and improved medical devices that provide greater features and functionality, and devices which generally help improve the lives of human beings and other animals.

One such area in which there is a need for new and improved medical devices is in the field of surgery. Surgery generally involves a physical intervention on tissues. A surgical procedure or operation may be done to investigate, explore, or treat a pathological condition such as disease or injury, to help improve bodily function, or to enhance physical appearance.

An anastomosis is a surgical procedure that refers to the joining of two pieces of tissue, such as connecting an artery to an artery, a vein to a vein, an artery to a vein or connecting the healthy sections of the colon or rectum after the diseased portion has been removed.

The microvascular anastomosis is a particular type of surgical procedure that refers to the joining of two blood vessel ends (e.g., end-to-end anastomosis) or joining an end of a blood vessel to the side of another blood vessel (e.g., end-to-side anastomosis).

Doctors use such procedures during, for example, bypass surgeries, removal of a diseased portion of a blood vessel, or free tissue transfer reconstructions. In a free tissue or free flap transfer reconstruction, the doctor detaches tissue from one site (i.e., donor site) on the body and transfers it to another location (i.e., recipient site) on the body. The procedure includes connecting the donor blood vessel ends with the recipient blood vessel ends. Various types of tissue may be transferred as free tissue including skin and fat, muscle, nerve, bone, or any combination of these. Doctors perform free tissue transfers for any number of reasons including cosmetic reconstruction, traumatic reconstruction, and removal of cancer from the mouth, jaw, or neck.

In many cases, the two vessel ends (i.e., donor vessel end and recipient vessel end) are too short to reach each other, their diameters are different, or both. Therefore, a vessel extender is needed.

### BRIEF SUMMARY OF THE INVENTION

An anastomotic device includes a tube and anastomotic coupler rings having vessel openings attached to each end of the tube. In an embodiment, the diameters of the vessel openings are the same.

In an embodiment, the device includes a first coupler ring, including a first plurality of pins on a first side, a first plurality of pin openings on a second side, opposite of the first side, and a first vessel opening having a first diameter, a second coupler ring, including a second plurality of pins on a third side, a second plurality of pin openings on a fourth side, opposite the third side, and a second vessel opening having a second diameter. The second diameter is the same as the first diameter. A tube, which includes an artificial material and graduated markings along a portion of the tube, is connected between the first and second vessel openings.

Furthermore, the tube may include a first band in a first color and a second band in a second color, where the second color is different from the first color, the first color indicates a length of the tube, and the second color indicates the first diameter.

The artificial material may include at least a polymer based material or at least a woven material.

In an embodiment, the tube includes a first coating on a first portion of an inner surface of the tube, where the first coating includes a first drug. The tube may include a second coating on a second portion of the inner surface of the tube, and the second coating may include a second drug, different from the first drug.

There may be a first jaw assembly holding the first coupler ring and a second jaw assembly holding the second coupler ring.

In an embodiment, a vessel extender kit includes a container, including a plurality of vessel extenders, where a first vessel extender includes a first coupler ring having a first vessel opening, a second coupler ring having a second vessel opening, and a first tube, connected between the first and second vessel openings. A second vessel extender includes a third coupler ring having a third vessel opening, a fourth coupler ring having a fourth vessel opening, and a second tube, connected between the third and fourth vessel openings. The first tube has a different length than the second tube.

The first tube may be treated with a cryoprotectant solution. Diameters of the first and second vessel openings may be the same. The diameter of the first vessel opening may be different from the diameter of the third vessel opening.

The container may further include a base member, a recloseable lid, and a tray that fits in the base member, the tray having a first cavity to hold the first vessel extender and a second cavity to hold the second vessel extender.

In an embodiment, in the tray, adjacent to the first cavity is a first label and adjacent to the second cavity is a second label, different from the first label. The first label may indicate a length of the first vessel extender and the second label may indicate a length of the second vessel extender. The first label may indicate a diameter of the first vessel extender and the first label may have the shape of a circle.

The vessel extender kit may further include at least one of an irreversible high temperature indicator or an irreversible low temperature indicator attached to a surface of the container. In a specific embodiment, the first tube of the vessel extender kit includes a natural material. The natural material may be a human allograft, a xenograft, or freeze-dried human tissue.

In an embodiment, a method includes providing a first coupler ring having a first vessel opening and a second coupler ring having a second vessel opening, providing a tube having a first end and a second end, where the tube includes an artificial material. The method further includes attaching the first end of the tube to the first coupler ring, attaching the second end of the tube to the second coupler ring, and sealing the tube, first coupler ring, and second coupler ring in a package.

The package may include a label attached to an exterior surface of the package, where the label includes information indicating a length of the tube and diameters of the first and second vessel openings.

Before sealing the tube, first coupler ring, and second coupler ring in the package, the method may further include attaching a first jaw assembly to the first coupler ring, and attaching a second jaw assembly to the second coupler ring.

In an embodiment, a package to preserve the sterility of contents in the package includes a vessel extender including a first coupler ring, including a first plurality of pins on a first side, a first plurality of pin openings on a second side, opposite of the first side, and a first vessel opening having a first diameter, a second coupler ring, comprising a second plurality of pins on a third side, a second plurality of pin openings on a fourth side, opposite the third side, and a second vessel opening having a second diameter, where the second diameter is the same as the first diameter, a tube, connected between the first and second vessel openings, where the tube includes a natural material.

The natural material may be a human vein, a human artery, a human allograft, a xenograft, or freeze-dried human tissue.

The device may further include a first jaw assembly holding the first coupler ring and a second jaw assembly holding the second coupler ring.

A specific embodiment includes at least one of an irreversible high temperature indicator or an irreversible low temperature indicator attached to a surface of the package.

In an embodiment, a device includes a first coupler ring, a second coupler ring, and a tube, including a first opening and a second opening, where the first coupler ring is connected to the first opening, the second coupler ring is connected to the second opening, and the tube is made from a natural material, and a first marking, visible from an outside of the tube, indicating a length of the tube. A second marking may indicate a date of manufacture of the device.

A stent may be included within a lumen of the tube. The embodiment may further include a first jaw assembly holding the first coupler ring and a second jaw assembly holding the second coupler ring. The tube may have been treated with a cryoprotectant solution.

Other objects, features, and advantages of the present invention will become apparent upon consideration of the following detailed description and the accompanying drawings, in which like reference designations represent like features throughout the figures.

### BRIEF DESCRIPTION OF THE DRAWINGS

FIG. 1 shows a perspective view of a first embodiment a vessel extender.

FIG. 2 shows a side view of a vessel extender with circumferential corrugations on an outer surface of the tube.

FIG. 3 shows a side view of a vessel extender with spiral corrugations on an outer surface of the tube.

FIG. 4 shows a side view of a second embodiment of a vessel extender that includes an integrated stent.

FIG. 5 shows a cross-sectional view of the vessel extender with a stent positioned between an inner and outer tube.

FIG. 6 shows a cross-sectional view of the vessel extender with a stent positioned within the lumen of the tube.

FIG. 7 shows a cross-sectional view of the vessel extender with a stent positioned around the tube.

FIG. 8 shows a side view of a third embodiment of a vessel extender having graduated markings.

FIG. 9 shows a cross-sectional view of the tube of the vessel extender.

FIG. 10 shows a front view of a coupler ring.

FIG. 11 shows a side view of the coupler ring.

FIG. 12 shows a perspective view of a second or back side of the vessel extender.

FIG. 13 shows a perspective view of the vessel extender joining with a body vessel.

FIG. 14 shows a perspective view of a fourth embodiment of a vessel extender with a coupler ring having six pins and six pin openings.

FIG. 15 shows a perspective view of a vessel extender with jaw assemblies holding the coupler rings.

FIG. 16 shows a perspective view of a specific implementation of an anastomotic coupler assembly tool being used to connect the vessel extender to a body vessel.

FIG. 17 shows a front view of a first implementation of a vessel extender kit.

FIG. 18 shows a front view of a second implementation of a vessel extender kit.

FIG. 19 shows a front view of a third implementation of a vessel extender kit.

FIG. 20 shows a flow diagram representative of a user using a vessel extender.

FIG. 21 shows a side view of a fifth embodiment of a vessel extender which may be referred to as a vessel right sizer.

FIG. 22 shows a side view of a vessel extender with an hourglass shape.

FIG. 23 shows a side view of a vessel extender with a bulged shape.

FIG. 24 shows a side view of a vessel right sizer that includes graduated markings.

FIG. 25 shows a front view of a vessel right sizer kit.

FIG. 26 shows a side view of a sixth embodiment of a vessel extender that includes a multibranch tube.

FIG. 27 shows a flow diagram representative of the steps to create a vessel extender or vessel right sizer.

FIG. 28 shows a side view of a vessel extender with a straight tube.

FIG. 29 shows a side view of a vessel extender with a tube with a triangle shape.

FIG. 30 shows a side view of a vessel extender with a tube with a serpentine shape.

FIG. 31 shows a side view of a vessel extender with a tube with a 90-degree elbow.

FIG. 32 shows a side view of a vessel extender with a tube with a 45-degree elbow.

FIG. 33 shows a side view of a vessel extender with a tube with an offset.

FIG. 34 shows a side view of a vessel extender with a tube with a U-shape.

FIG. 35 shows a side view of a vessel extender with a tube with a T-shape.

FIG. 36 shows a side view of a vessel extender with a tube with a Y-shape.

FIG. 37 shows a side view of a vessel extender with a multibranch tube.

FIG. 38 shows a side view of a vessel extender with a branch of the multibranch tube cut off and closed shut.

FIG. 39 shows a side view of a vessel extender with a tube with square shaped corrugations.

FIG. 40 shows a side view of a vessel extender with a tube with triangle shaped corrugations.

FIG. 41 shows a side view of a vessel extender with a tube with sawtooth shaped corrugations.

FIG. 42 shows a side view of a vessel extender with a tube with staircase shaped corrugations.

FIG. 43 shows a side view of a vessel extender with a tube portion having corrugations and another tube portion that is smooth.

FIG. 44 shows a side view of a vessel extender with a tube portion having corrugations between smooth tube portions.

FIG. 45 shows a side view of a vessel extender with a tube portion that is smooth between corrugated tube portions.

FIG. 46 shows a side view of a vessel extender with tube portions that have different types of corrugations (e.g., square shaped corrugations and triangle shaped corrugations).

### DETAILED DESCRIPTION OF THE INVENTION

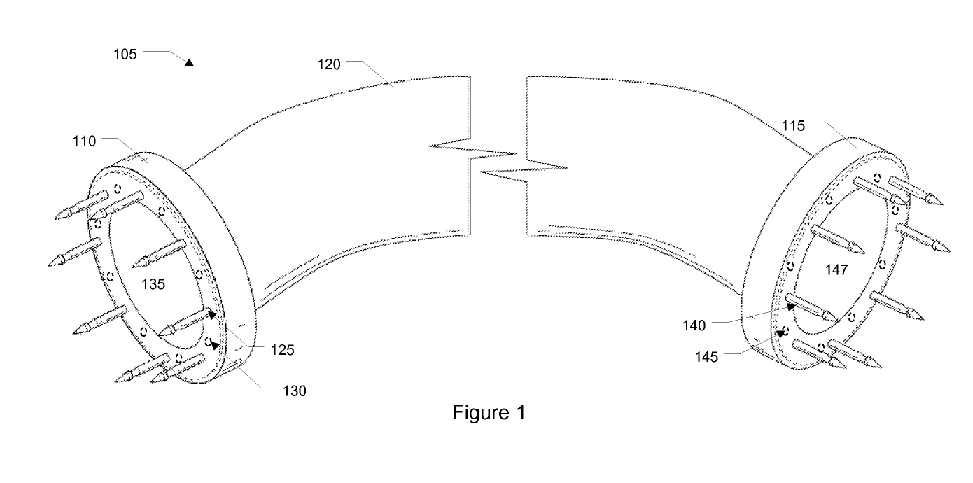

FIG. 1 shows a perspective view of a first embodiment of a vessel extender 105, which may be referred to as an “artificial vessel extender.” The vessel extender includes a first coupler ring 110, a second coupler ring 115, and a tube 120 between the first and second coupler rings. The first coupler ring includes a first set of pins 125, a first set of pin openings 130, and a first vessel opening 135. The second coupler ring includes a second set of pins 140, a second set of pin openings 145, and a second vessel opening 147. The tube is connected between the first and second vessel openings. In this first embodiment of the vessel extender, the diameters of the first and second vessel openings are the same.

The vessel extender is typically used during an anastomosis. An anastomosis is a surgical procedure that refers to the joining or connecting of two pieces of tissue. This includes connecting two body vessels (e.g., blood vessels), connecting the healthy sections of the colon or rectum after the diseased portion has been removed, or connecting the stomach to the jejunum (e.g., gastrojejunal anastomosis).

If, for example, the two pieces of tissue or body vessels (e.g., first and second blood vessels or donor and recipient blood vessels) are too short to reach each other then the vessel extender can be placed between the donor and recipient blood vessels to bridge the gap between them and thus join the two blood vessels together via the coupler rings. For example, a third coupler ring is connected to the donor vessel (or first body vessel) by passing the donor vessel through the vessel opening of the coupler ring, everting the donor vessel over the pins, and impaling the donor vessel onto the pins. The third coupler ring is then joined to the first coupler ring of the vessel extender. That is, the pins of the third coupler ring mate with the pin openings of the first coupler ring. Conversely, the pins of the first coupler ring mate with the pin openings of the third coupler ring.

A similar procedure is followed at the opposite end of the vessel extender. That is, a fourth coupler ring is connected to the recipient blood vessel (or second body vessel). The fourth coupler ring then mates with the second coupler ring of the vessel extender.

In various implementations, the tube is made from artificial materials, man-made materials, natural materials, or combinations of these. Some examples of these materials include: polymers, synthetic and natural fibers, materials formed from polymers, composite materials of at least one polymer and at least one nonpolymer, woven materials, ceramics, products of cellular synthesis, products of cellular reproduction, or combinations of these.

Woven materials may have any type of weave. Some examples of weaves include plain weaves (i.e., tabby weave or taffeta weave), rib weaves, balanced plain weaves, basket weaves, regular basket weaves, irregular basket weaves, satin weaves, twill weaves, leno weaves, mock leno weaves, and gauze weaves.

Polymers include polyurethaneurea, polyethylene terephthalate, polypropylene polyester, polytetrafluoroethene, polycarbonate, and silicone rubber. Synthetic and natural fibers include polylactic acid, aramid fibers, vinylidene chloride, polyphenylene sulfide, nylon, polyvinyl chloride fibers (i.e., vinyon), and cellulose acetate. Materials formed from polymers include fiberglass made from the extrusion of silica, carbon fibers obtained from pyrolysis of polyacrylonitrile, and latex comprising an emulsion of polymer microparticles in an aqueous medium.

Composite materials include polyethylene with carbon nanotubes to strengthen the polyethylene. Tube materials may be produced by growing cloned vascular cells on a polyethylene terephthalate lining, and removing vascular cells from a patient to grow on a polypropylene mesh.

Materials using products of cellular synthesis or cellular reproduction include a tube of collagen fibers on a polyurethane mesh, a vascular autograft taken from a patient and used in another part of the same patient, a vascular allograft taken from another organism of the same species (e.g., human cadavers) that may be fitted with a biodegradable mesh, and a vascular xenograft or xerograft taken from a different species (e.g., pigs) that may be treated with supercritical carbon.

In a specific embodiment, the tube is derived from cells (e.g., human cells, pig cells, and rabbit cells). These donor cells are used to create a collagen matrix. The cells are then removed so that the final material of the tube is a cellular. One benefit is that such a tube typically has a longer shelf life as compared to tubes with cellular products. This can also help to reduce the storage and shipping costs for the vessel extender since the tube does not include cellular material.

Examples of natural materials include tissue (e.g., human tissue), human veins, human arteries, human allografts (e.g., blood vessel from a human cadaver), or xenografts (e.g., blood vessel from a pig, cow, rabbit, ape, monkey, or horse).

In a specific implementation, the material includes a human allograft vessel. The human allograft vessel is processed to help prevent, for example, infectious disease contamination, cross contamination, or both. The inner surface of the processed human allograft vessel may be altered to produce one or more desirable properties or effects, such as reducing undesirable turbulence and eddy formation at the vessel wall and blood interface. Altering the inner surface is optional and some implementations do not include altering the inner surface. In a specific embodiment, the processed human allograft includes a biodegradable mesh. In another embodiment, the biodegradable mesh is omitted.

In specific implementations where a natural material is included the natural material is typically processed so that it can be preserved for storage and shipping. In a specific embodiment, the natural material (i.e., tissue) is freeze-dried. The freeze-drying process generally includes treating the tissue with a cryoprotectant solution to protect the tissue from damage (e.g., damage due to ice formation). The cryoprotectant solution may include glycols, propylene glycol, dimethyl sulfoxide, an amide such as formamide, urea, acetamide, hydroxyurea, N-methyl formamide, and ethylene glycol or ethylene glycol in combination with propylene glycol, mannitol, water, sucrose, sodium phosphate, or trehalose.

In a specific implementation, the material includes Gore-Tex. Gore-Tex is a registered trademark of W.L. Gore & Associates, Incorporated of Newark, Del. In this specific implementation, the material includes a porous form of polytetrafluoroethylene with a microstructure characterized by nodes interconnected by fibrils. The material is based on thermomechanically expanded polytetrafluoroethylene (PTFE) and other fluoropolymer products.

Typically, the tube has properties that are similar to the desirable properties of the body vessels that it joins. For example, like body vessels, the tube is generally flexible, soft, and pliable so that the tube can be routed around other tissues in the body cavity as needed.

The tube is manufactured as a generally straight member. However, in other implementations, the tube includes one or more bends, angles, turns (e.g., U-turns), or elbows (e.g., 15-degree elbow, 30-degree elbow, 45-degree elbow, 60-degree elbow, 90-degree elbow, and 135-degree elbow). These elbows may be used to help route the tube around other tissues so that the tube does not become kinked or crushed if the routing of the tube includes sharp turns.

In a specific embodiment, the tube includes a material (e.g., shape-memory material) that retains its shape after being bent. The material includes shape-memory polymers, metals, or both. After the tube is bent around other tissues in the body cavity, heat, light, chemicals, or combinations of these may be applied to the tube to trigger a reaction that causes the tube to retain its new shape.

Typically, the tube has a stiffness value such that other tissues (e.g., organs) surrounding the tube will not be able to squeeze the tube or cause an undesirable deflection in the tube that disrupts blood flow. However, the tube is also typically bendable so that it can be routed around other tissues in the body cavity. For example, depending on the application, the user can bend the vessel extender into any angle between 0 degrees and 180 degrees (e.g., bend into a U-turn) without the vessel extender collapsing.

The stiffness of the tube and ability of the tube to bend without collapsing is due, in part, to the shape of the tube. For example, generally, a tube with a corrugated surface has a greater circumferential or hoop stiffness than a tube without a corrugated surface. Such a tube also typically has a greater tolerance to bending before buckling.

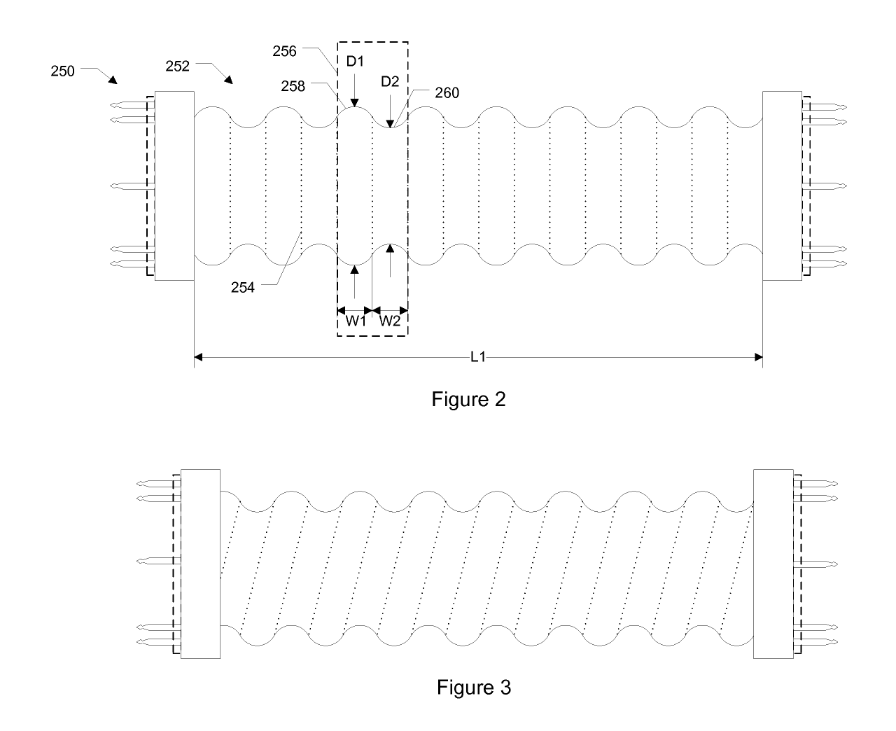

For example, FIGS. 2 and 3 each show a side view of a specific implementation of a vessel extender with a tube having a corrugated surface. FIG. 2 shows a tube having circumferential corrugations. FIG. 3 shows a tube having spiral corrugations. The corrugated surface allows the tube to bend, turn, and generally conform to the particular routing within the body cavity without collapsing.

In FIG. 2, a vessel extender 250 includes a tube 252 with a corrugated surface 254 or multiple projections and recesses or ridges and grooves which at least partially surround the tube. The corrugated surface may be defined by one or more units 256 which are typically repeated along at least a portion of a total length L1 of the tube. Each unit typically includes at least one projection and at least one recess or, in implementations where the tube has a circular cross section—a portion with a larger diameter adjacent to another portion with a smaller diameter.

For example, in a specific implementation, a projection 258 has a diameter D1 and a width W1. A recess 260 has a diameter D2 and a width W2. Diameter D1 is different from diameter D2. Typically, diameter D1 is greater than diameter D2. For example, diameter D1 may be about 10, 20, 30, 40, 50, 100, 200, 300, 400, 500, 600 percent, or more than 600 percent greater than diameter D2. In a specific implementation, diameter D1 is less than 10 percent greater than diameter D2.

A ratio of the smaller diameter (i.e., diameter D2) to the larger diameter (i.e., diameter D1) is typically in the range of about 1:1.01 to about 1:6.5.

Diameters D1 and D2 range from about 0.5 millimeters to about 12 millimeters. Some examples include 0.7, 0.9, 1.5, 2, 2.5, 3, 3.5, 4, 4.5, 5, 5.5, 6, 6.5, 7, 7.5, 8, 8.5, 9, 9.5, 10, 10.5, 11, 11.5, or 11.9 millimeters. Depending upon the application, diameter D1, diameter D2, or both may be less than 0.5 millimeters or greater than 12 millimeters.

In a specific implementation, widths W1 and W2 are the same. In another implementation, width W1 is different from width W2. That is, width W1 may be greater than width W2. Width W1 may be about 10, 20, 30, 40, 50, 100, 200, 300, 400, 500, 600 percent, or more than 600 percent greater than width W2. In a specific implementation, width W1 is less than 10 percent greater than width W2.

In another implementation, width W2 is greater than width W1 Width W2 may be about 10, 20, 30, 40, 50, 100, 200, 300, 400, 500, 600 percent, or more than 600 percent greater than width W1. In a specific implementation, width W2 is less than 10 percent greater than width W1.

Widths W1 and W2 range from about 0.4 millimeters to about 5 millimeters. Some examples include 0.5, 0.7, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, or 4.9 millimeters. Depending upon the application, width W1, width W2, or both, may be less than 0.4 millimeters or greater than 5 millimeters.

Generally, a unit is repeated along at least a portion of the total length (L1) of the tube. The number of units repeating along a length L2 of the tube is represented by the following equation:

$$number of units repeating = \frac{L 2}{W 1 + W 2} , L 2 \leq L 1 \qquad ( 1 )$$

In various implementations, a unit is repeated continuously along at least a portion of the tube, but is interrupted by a smooth portion of tube. For example, a first portion of the tube may include one or more units repeating. A second portion of the tube may be smooth (FIG. 43).

As another example, one or more units may be between smooth portions of the tube. That is, first and second portions of the tube may be smooth. A third portion of the tube, between the first and second portions of the tube, may include one or more units repeating. (see FIG. 44).

Likewise, one or more units may be on opposite ends of a smooth portion of the tube. For example, a first portion of the tube may include one or more units repeating. A second portion of the tube may include one or more units repeating. A third portion of the tube, between the first and second portions of the tube, may be smooth (FIG. 45).

This feature of the tube allows the smooth portion of the tube to be used for vessel extender routing segments that are straight. The portion of the tube having the corrugated surface (i.e., projections and recesses) can be used for routing segments that include bends.

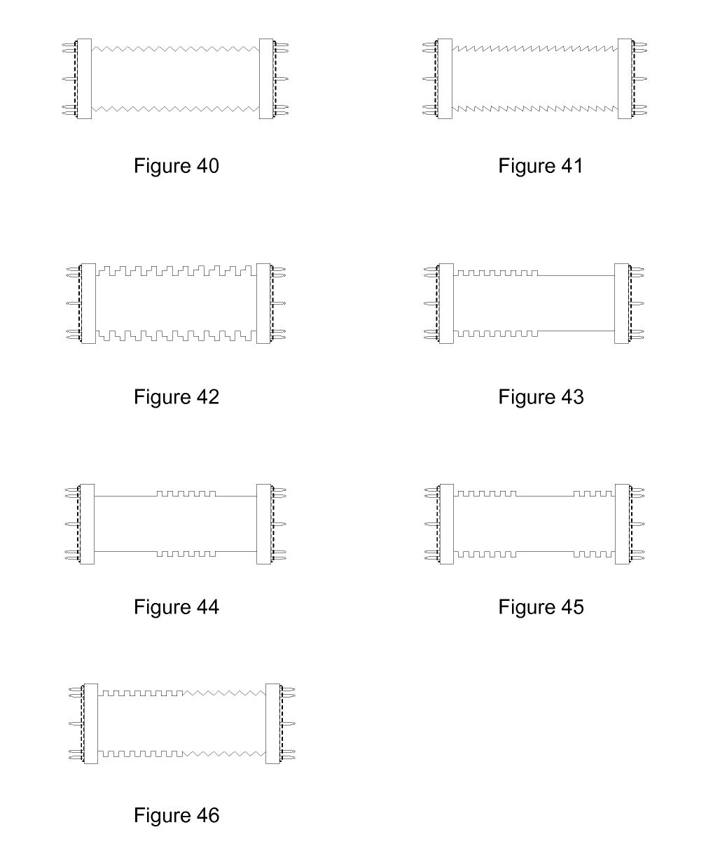

The corrugations (i.e., projections and recesses) may have any shape. For example, in the example of FIG. 2, the projections and recesses are rounded or have a sine waveform. However, in other implementations, the projections and recesses have, for example, square shapes (e.g., square waveform, FIG. 39), triangle shapes (e.g., triangle waveform, FIG. 40), sawtooth shapes (e.g., sawtooth waveform, FIG. 41), or staircase shapes (e.g., staircase waveform, FIG. 42).

In the example shown in FIG. 2, the unit includes one projection and one recess. However, it should be appreciated that a unit may include any number of projections and any number recesses.

Furthermore, although FIG. 2 shows the corrugations on the outer surface of the tube, it should be appreciated that the corrugations may instead or additionally be located on an inner surface of the tube. Generally, however, the inner surface of the tube is smooth to help prevent blood from clotting on the inner surface.

In a specific implementation, a tube includes projections and recesses with varying shapes. For example, a first portion of the tube may include units with projections and recesses that have square shapes. A second portion of the tube may include units with projections and recesses that have triangle shapes (FIG. 46).

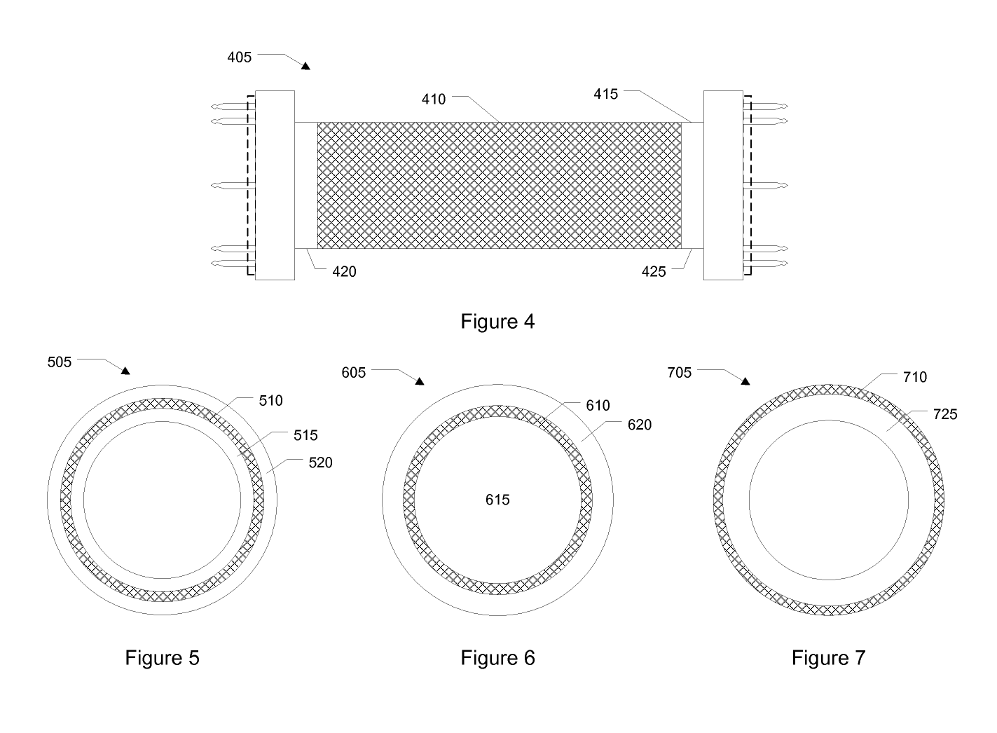

FIG. 4 shows a second embodiment of a vessel extender 405 that includes a stent 410 that is integrated or built into a tube 415 of the vessel extender. The stent runs along at least a portion of the length of the tube. In a specific embodiment, the stent is between first and second ends 420 and 425 of the tube as shown in the example of FIG. 4. In another embodiment, the stent extends past the first end of the tube, the second end of the tube, or both.

The stent can be used to increase the rigidity of the tube and thus make the tube more rigid than a tube without the stent. Typically, the stent is also bendable so that it can change shape. This allows the stent to fit and follow the routing of the vessel extender in the body cavity. Generally, the stent also has stiffness such that it can resist the forces of other tissues (e.g., organs) pressing on it. The stent helps to prevent the tube from collapsing and obstructing the flow of blood to the tissues. For example, the stent may help to prevent fibrosis (i.e., development of excess fibrous connective tissue), the formation of a thrombus (i.e., blood clot), or stenosis (i.e., abnormal narrowing).

The stent may be made of metal or polymers (e.g., silicone, plastic, polyurethane, and shape-memory polymers). Some examples of metals include chrome, nickel, steel (e.g., 316L stainless steel), gold, titanium, niobium, platinum, zirconium, a cobalt-chromium alloy, a tantalum alloy, magnesium, or nickel titanium (i.e., nitinol). The stent may include one or more coatings such as an iridium oxide coating to enhance the biocompatibility and antiproliferative characteristics of the stent. The thickness of the coating ranges from about 10 nanometers to about 1000 nanometers or from about 5 microns to about 30 microns, including less than 5 microns and more than 30 microns.

Various embodiments of the vessel extender include a stent including biodegradable materials. Biodegradable materials include biodegradable polymers, copolymers, block polymers, and combinations of these. Such stents are typically used in short-term or temporary applications. For example, a biodegradable stent may be designed to degrade and be absorbed by the body within about three or four months. One benefit of such stents is that they reduce any long-term problems associated with exposing foreign materials to the blood (e.g., allergic reactions).

Some examples of stents that may be included with the tube include coronary stents, bare metal stents, drug-eluting stents (i.e., drug coated stents), covered stents, uncovered stents, ureteral stents, vascular stents, peripheral vascular stents, stent grafts, coronary heat disease (CHD) stents, rectal stents, oesophageal stents, biliary stents, pancreatic stents, and stents combined with a blood vessel graft.

The stent is typically attached to the tube with an adhesive (i.e., glue) such as a polymer gel (e.g., cyanoacrylate polymer), silicone adhesive, or by using any other type of biocompatible adhesive.

FIGS. 5-7 show cross-sectional views of the tube and stent in various embodiments.

FIG. 5 shows a cross-sectional view of a first embodiment a vessel extender with a built-in stent. A vessel extender 505 includes a stent 510 positioned between an inner tube 515 and an outer tube 520. That is, the stent is sandwiched between the inner and outer tubes.

FIG. 6 shows a cross-sectional view of a second embodiment of a vessel extender with a built-in stent. A vessel extender 605 includes a stent 610 positioned within or inside a lumen 615 of a tube 620.

FIG. 7 shows a cross-sectional view of a third embodiment of a vessel extender with a built-in stent. A vessel extender 705 includes a stent 710 positioned outside a tube 725. The stent surrounds at least a portion of the length, circumference, or both of the tube.

Various implementations of the vessel extender include two or more stents that may be in any configuration. In a specific implementation, a first stent is sandwiched between an inner and outer tube as shown in FIG. 5. A second stent at least partially surrounds a portion of the tube as shown in FIG. 7. The two stents serve different purposes. The first stent is used to help prevent the vessel extender from collapsing or crimping. The second stent includes a drug coating. Over a period of time, the drug diffuses through the tube and is then absorbed by the blood flowing through the vessel extender.

Some embodiments of the vessel extender include only a single coupler ring attached to an end of the tube while the opposite end of the tube is unconnected to a coupler ring. This specific embodiment of the vessel extender is used in situations where, for example, the doctor has difficulty everting one of the two body vessels over the pins of the third coupler ring to which the vessel extender will join. For example, the body vessel may have unusually thick walls which prevent the body vessel from being turned outwards and onto the pins of the third coupler ring. In this case, the doctor may choose to attach the body vessel to the tube using other means such as gluing, stitching, or suturing the body vessel to the tube.

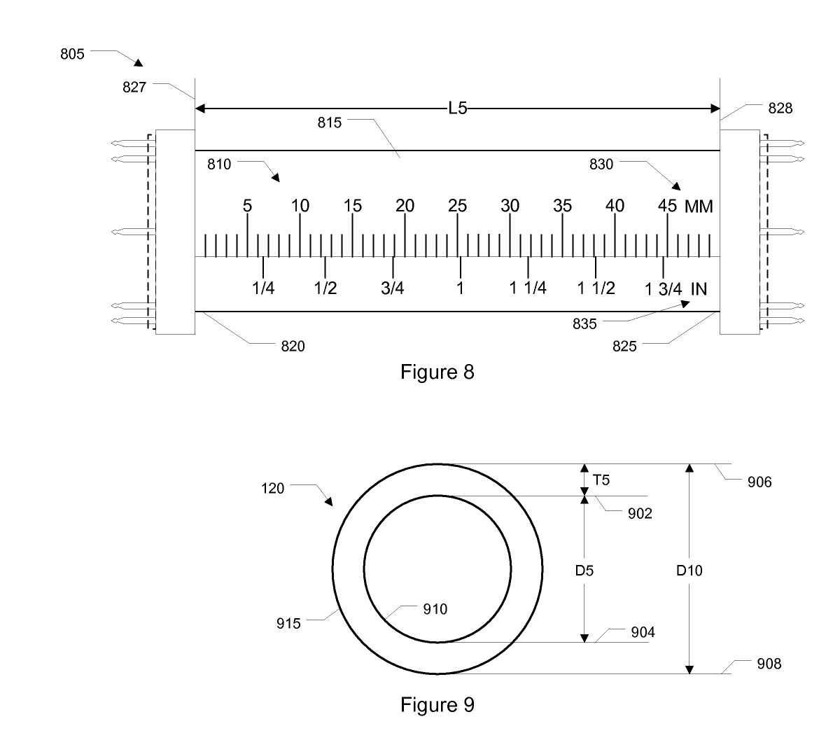

FIG. 8 shows a side view of a third embodiment of a vessel extender 805. Vessel extender 805 includes a set of vertical lines or graduated markings 810 on a tube 815 which span from a first end 820 of the tube to a second end 825 of the tube. A distance L5 (i.e., tube length) is between lines 827 and 828.

The graduated markings may be printed markings. In a specific embodiment, the graduated markings correspond to one or more units of measurement and include numbers as shown in the example of FIG. 8. That is, they can resemble a ruler or measuring tape to indicate length. The graduated markings show a distance from the first end of the tube to the second end of the tube. In the example shown in FIG. 8, a first set of graduated markings 830 is in millimeters and a second set of graduated markings 835 is in inches. However, it should be appreciated that other units of measurements such as centimeters may be used.

A specific embodiment includes multiple sets of graduated markings evenly distributed around or about the tube. In this specific embodiment, a third set of graduated markings is on a side of the tube opposite the first set of graduated markings. The third set of graduated markings is the same as the first set of graduated markings. This feature helps to ensure that the user can see the graduated markings without having to flip the vessel extender.

In a specific implementation, the vessel extender is designed to be cut to length. That is, after the user or doctor determines the length needed to join two body vessels, the doctor can use the graduated markings to measure the length of vessel extender needed and cut the tube. Because the graduated markings are on the tube, the doctor does not have to use a separate ruler or measuring tape to measure the length of tube to cut.

The doctor can then attach a new coupler ring to the cut end of the tube by passing the cut end of the tube through the new coupler ring, everting the tube over the pins, and impaling the tube onto the pins of the new coupler ring.

The vertical lines or graduated markings may be in different colors to help the user see the markings. Generally, the graduated markings are in a color which is different from the color of the tube. Similarly, the first set of graduated markings may be in a first color, the second set of graduated markings may be in a second color, different from the first color. This can help the user distinguish between the two units of measurements.

In a specific embodiment, the markings do not correspond to any specific unit of measurement. Instead, the markings indicate one or more positions or reference points on the tube between the first and second end of the tube. The markings are separated by one or more regular or irregular intervals. Each interval may be identified with, for example, a number, symbol, character, letter, or the like.

In this specific embodiment, the doctor determines the length of a vessel extender needed by holding together the first end of the vessel extender and first body vessel. The first end of the vessel extender and the first body vessel may be held together using clamps (e.g., vascular clamps). The doctor then routes the second end of the vessel extender towards the second body vessel and arranges the second body vessel such that it overlaps the second end of the vessel extender and at least a portion of the tube. The end of the overlap provides an indication of the position at which the tube should be cut to size. With the markings, the doctor can note the marking which corresponds to the end of the overlap and cut the tube at or near that marking.

In this specific embodiment, the markings include lines along at least a portion of the tube. Typically, the lines are positioned transversely to a longitudinal axis of the tube. Numbers adjacent to the lines are optional and are omitted in some implementations. In a specific embodiment, a line encircles the tube. That is, the length of the line is the same as the circumference of the tube. In another embodiment, a line at least partially encircles the tube. That is, the length of the line may be less than the circumference of the tube. For example, the length of the line may be the same as or less than the diameter of the tube. The length of the line may be the same as or less than the radius of the tube. A ratio of the length of the line to the radius of the tube typically ranges from about 1:1 to about 1:10. Some examples of the ratio include 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, or 1:9.9.

In a first embodiment, the length of each line is the same and the lines are equally spaced from each other. In a second embodiment, two lines have different lengths, the lines are equally spaced from each other, and the number of lines with shorter lengths is greater than the number of lines with longer lengths. In this second embodiment, at least two lines (e.g., four lines) with shorter lengths are between two lines with longer lengths. It should be appreciated that there may be any number of lines. The lines may be equally or unequally spaced from each other and have any number of different lengths.

Typically, the lines indicate a reference point or a length measurement from an end of the tube. However, one or more lines may instead or additionally indicate a different measurement such as the diameter of the tube or the thickness of the tube.

The tube length (L5) ranges from about 20 millimeters to about 200 millimeters. Some examples of tube lengths include 25, 30, 35, 40, 45, 50, 55, 60, 65, 70, 75, 80, 85, 90, 95, 100, 110, 120, 130, 140, 150, 160, 170, 180, 190, or 199 millimeters. Depending upon the application, the tube length may be less than 20 millimeters or greater than 200 millimeters.

In various embodiments, the vessel extender includes one or more visual indicators or markings to indicate some property or characteristic of the vessel extender. In a specific embodiment, the markings are in the form of a barcode or serial number. The barcode or serial number is used as an index to a record in a database. The record includes information, such as the properties and characteristics of the vessel extender and patient information.

Such markings may be made using any technique for making a visible impression on the vessel extender including, but not limited to, printing, silkscreen printing, masking, stamping, plating, thermography, embossing, painting, engraving, etching, anodizing, oxidizing, deposition, imprinting, and chemical processing. The marking may be made anywhere on the extender such as the coupler ring, outside of the tube, or inside of the tube. However, it is generally desirable that the marking is visible from an outside of the vessel tubing.

The markings may include a chemical, dye, or contrast substance that is visible during a fluoroscopy. This allows the location of the vessel extender in the patient to be identified during a fluoroscopy. Identifying the location of the vessel extender in the patient can be used to help guide future surgical procedures. This also allows gathering information about the vessel extender (e.g., length, diameter, manufacturing date, and serial number) without having to resort to exploratory surgery or to the patient's medical file which may be incomplete or missing.

In a specific embodiment, the markings are designed to be permanent so that if the patient undergoes another surgical procedure at the same surgical site at a later date, the markings will still be visible. In another embodiment, the markings are not permanent. The markings may be made of a material that can dissolve and be absorbed by the body. For example, the markings may be heat activated such that the markings will degrade after a threshold temperature is reached. As another example, the markings may be included on an adhesive strip that is attached to the tube and that can be removed by the doctor.

Thus, as part of the implantation process, the doctor can record the serial number of the vessel extender implanted in the patient. This allows the vessel extender to be tracked and cross-referenced to the patient. If any contraindications are later identified the patient's doctor or some other entity can notify the patient by cross-referencing in the database the serial number of the vessel extender to the patient.

Table A below lists some examples of information that may be included on the vessel extender, cross-referenced to a database, or both using the visual indicators or markings on the vessel extender.

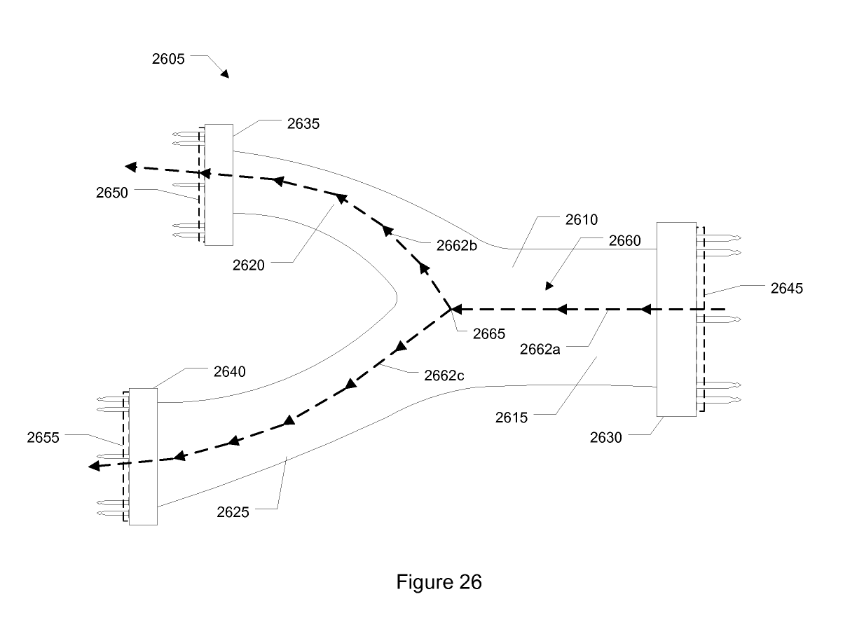

TABLE A

| | |
|---|---|
| Length of vessel extender | Country of origin (e.g., U.S.A., China, |
| | Taiwan, India, Brazil, France, Italy, and |
| | Canada) |
| Diameter of vessel extender (e.g., tube outer | Name, mailing address, phone number, and e- |
| diameter, tube inner diameter, and coupler | mail address of manufacturer, distributor, and |
| ring vessel opening diameter) | any other entity involved in the distribution of |
| | the vessel extender |
| An angle between any two tubing branches | Name, mailing address, phone number, and e- |
| (see FIG. 26) | mail address of the doctor who performed the |
| | implant |
| Tube material | Name, mailing address, phone number, and e- |
| | mail address of the doctor regularly following |
| | the patient |
| Tube compliance and burst pressure | Name, mailing address, phone number, e-mail |
| | address, and social security number of patient |
| Whether the vessel extender includes a stent | Date vessel extender was implanted |
| and the type of stent | |
| Whether the vessel extender includes a drug | Lot number, model number, batch number, |
| coating and the type of drug coating | serial number, or other identifier for the vessel |
| | extender |
| Date of manufacture | Date of shipment (e.g., date of shipment from |
| | manufacturer, date of shipment from |
| | distributor) |
| Expiration date | Instructions for use (e.g., implantation |
| | procedures) |
| Shelf life (e.g., X years, Y months) | Body vessel diameter (or range of body vessel |
| | diameters) that the vessel extender can be |
| | attached to |

The visual indicators may be color indicators, letters, words, numbers, characters, symbols, icons, graphic images, graphics, pictures, bumps, indentations, patterns, logotypes, trademarks, or the like.

In a specific embodiment, the tube includes one or more bands in one or more colors which indicate various properties or characteristics of the vessel extender. A color legend is typically included so that the user can match the color of the band with a description of what the color indicates. A first band in a first color indicates the length of the tube. For example, a first band colored red may indicate a tube with a length of 10 millimeters. A first band colored blue may indicate a tube with a length of 20 millimeters, and so forth.

A second band, next to the first band, in a second color indicates the diameter of the vessel opening of the coupler ring. For example, a second band colored green may indicate a vessel opening with a diameter of 1.5 millimeters. A second band colored purple may indicate a vessel opening with a diameter of 2.0 millimeters, and so forth.

A third band, next to the second band, in a third color indicates whether the tube has a drug coating and type of drug coating. For example, a third band colored orange may indicate that the tube does not have a drug coating. A third band colored black may indicate that the tube has an anticoagulant drug coating, a third band colored yellow may indicate that the tube has an antiplatelet drug coating, and so forth.

In a specific embodiment, a visual indicator includes one or more lines (e.g., printed lines) which span from the first end of the tube to the second end of the tube and are parallel to a longitudinal axis of the tube. The lines provide a visual indication to the doctor on whether the vessel extender is being twisted during implantation. Twists in the tube are generally undesirable because the twist may crimp the tube and obstruct the flow of the blood. If the doctor notices during implantation or after implantation that the lines are no longer parallel and are starting to twist, the doctor can correct the rotation of one of the coupler rings on the vessel extender to remove the twist from the tube.

FIG. 9 shows a cross-sectional view of tube 120. A distance D5 (i.e., tube inner diameter) is between lines 902 and 904. A distance D10 (i.e., tube outer diameter) is between lines 906 and 908. A distance T5 (i.e., tube thickness) is between lines 902 and 906.

Inner diameter D5 of the tube ranges from about 0.5 millimeters to about 12 millimeters. Some examples include 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 11.9 millimeters. Depending upon the application, the inner diameter of the tube is less than 0.5 millimeters or greater than 12 millimeters. The outer diameter D10 of the tube ranges from about 0.6 millimeters to about 14 millimeters. Some examples include 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, or 13.9 millimeters. Depending upon the application, the outer diameter of the tube is less than 0.5 millimeters or greater than 14 millimeters.

The variations in the inner and outer diameter of the tube reflect the many different diameters that a body vessel, such as a blood vessel can have. For example, arteries can have diameters of about 25 millimeters while capillaries can have diameters of about 8 microns. Generally, it will be desirable to select a vessel extender that has a tube with inner and outer diameters that match or are similar to the inner and outer diameters of the body vessel to which the tube will be connected.

The cross section of the tube can have any shape. Although FIG. 9 shows the tube having a circular cross section, other embodiments include a tube with, for example, a cross section that has the shape of an oval, ellipse, rectangle, square, octagon, triangle, or combinations of these.

Thickness T5 of the tube ranges from about 0.1 millimeters to about 3 millimeters. Some example thicknesses include 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 millimeters, or more than 2.9 millimeters. In some implementations, the thickness is less than 0.1 millimeters. The variation in the thickness of the tube reflects the many different thicknesses that a body vessel can have. For example, veins which carry blood back to the heart typically have thinner walls than arteries which carry blood away from the heart. Thus, in applications where the vessel extender connects two veins the tube thickness will generally be less than applications in which the vessel extender connects two arteries.

The thickness of the tube is generally inversely proportional to the compliance of the tube. Thicker tubes are generally less compliant than thinner tubes. Compliance is a measure of the tendency of body vessels such as arteries and veins to stretch in response to pressure. Compliance is calculated as a change in volume divided by a change in pressure. The compliance of the tube of the vessel extender will vary greatly depending upon the application. For example, a first implementation of the tube may have a compliance that is about 10 to about 20 times greater than the compliance of a second implementation of the tube. The variation in compliance reflects the different compliance measurements for body vessels (e.g., veins and arteries) at different pressures. Generally, it will be desirable to select a vessel extender that has a compliance that is similar to the two body vessels that the vessel extender will connect.

In various embodiments, the tube includes one or more coatings (e.g., drug coating) on an inner surface 910, an outer surface 915, or both. The thickness of the drug coating ranges from about 10 microns thick to about 50 microns thick, including less than 10 microns thick and greater than 50 microns thick. In a specific embodiment, the thickness of the drug coating is constant along the length of the tube. In another embodiment, the thickness of the drug coating varies along the length of the tube, circumference of the tube, or both.

For example, a first portion of the inner surface of the tube includes a first coating having a first thickness and a first drug. A second portion of the inner surface of the tube includes a second coating having a second thickness and a second drug. The first thickness is different from the second thickness. The different thicknesses allow, for example, releasing different amounts of drug into the blood. The first and second coatings may be adjacent to each other, some distance away from each other (e.g., separated by a portion of the tube having a third drug coating, separated by a portion of the tube not having any drug coating), or at least partially overlapping each other.

The first drug may be different from the second drug. As an example, the first drug may have a higher concentration or dosage of an active ingredient than the second drug, the active ingredient of the first drug may be different from the active ingredient of the second drug, the release rate of the first drug may be different from the release rate of the second drug, or combinations of these. Additionally, the first and second coatings may have different or the same surface areas.

In a specific embodiment, the coating is 30 microns thick and includes the drug paclitaxel in a layer of a polymer (e.g., poly(styrene-b-isobutylene-b-styrene)). Paclitaxel is sometimes used to help treat patients with cancer.

In another embodiment, there is a first coating which at least partially covers the inner surface and a second coating which at least partially covers the outer surface. The first coating is about 15 microns thick and includes the chemical compound nitric oxide embedded in a polymer. Nitric oxide can help to protect an organ such as the liver from ischemic damage. The second coating is 50 microns thick and includes the polymer poly[bis(trifluoroethoxy)phosphazene]. The tube then includes a material (e.g., porous material) that allows the drug in the second coating to diffuse from the outer surface of the tube and to the inner surface of the tube where the drug can be absorbed by the blood flowing through the tube.

The drug can include any substance or combination of substances capable of providing a therapeutic or prophylactic effect. For example, the drug can be used to help heal the anastomotic site. Examples of drugs include antiproliferative substances such as actinomycin D, or derivatives and analogs. Synonyms of actinomycin D include dactinomycin, actinomycin IV, actinomycin I₁, actinomycin X₁, and actinomycin C₁.

The drug can also fall under the genus of antineoplastic, antiinflammatory, antiplatelet, anticoagulant, antifibrin, antithrombin, antimitotic, antibiotic, antiallergic and antioxidant substances. Examples of such antineoplastics and antimitotics include paclitaxel, docetaxel, methotrexate, azathioprine, vincristine, vinblastine, fluorouracil, doxorubicin hydrochloride, and mitomycin. Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as bivalirudin.

Examples of such cytostatic or antiproliferative agents include angiopeptin, angiotensin converting enzyme inhibitors such as captopril, cilazapril or lisinopril, calcium channel blockers (such as nifedipine), colchicine, fibroblast growth factor (FGF) antagonists, fish oil (omega 3-fatty acid), histamine antagonists, lovastatin, monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium.

Other therapeutic substances or agents include alpha-interferon, genetically engineered epithelial cells, tacrolimus, dexamethasone, and rapamycin and structural derivatives or functional analogs thereof, such as 40-O-(2-hydroxy)ethyl-rapamycin, 40-O-(3-hydroxy)propyl-rapamycin, 40-O-[2-(2-hydroxy)ethoxy]ethyl-rapamycin, and 40-O-tetrazole-rapamycin.

In a specific embodiment, the coating includes phosphorescent chemicals to help detect any leaks in the vessel extender. After implantation, the doctor can shine a light (e.g., ultraviolet light) on the vessel extender. If any blood has leaked the blood shows up as a shadow or dark spot on the vessel extender. The doctor can then replace the vessel extender with a new vessel extender. This helps to ensure that the patient does not receive a damaged or improperly implanted vessel extender.

Similarly, in another embodiment, a coating including phosphorescent chemicals is applied to the coupler rings. This feature can help detect any gaps between joined coupler rings. After the doctor joins the coupler rings together, the doctor can shine the light on the coupler rings to see if any blood has leaked between the joined coupler rings. If any shadows or dark spots are seen then this may indicate a gap between the joined coupler rings through which the blood is leaking. The doctor can then close the gap by, for example, applying more force to squeeze the coupler rings together.

In a specific implementation, at least a portion of the inner surface of the tube, outer surface of the tube, or both include one or more surface features such as ridges, bumps, protrusions, grooves, or dimples. These surface features increase the surface area of the tube as compared to tubes with generally smooth surfaces. The increased surface area allows, for example, depositing a greater amount of a coating (e.g., drug coating) on the tube than would be the case if the tube had a smooth surface.

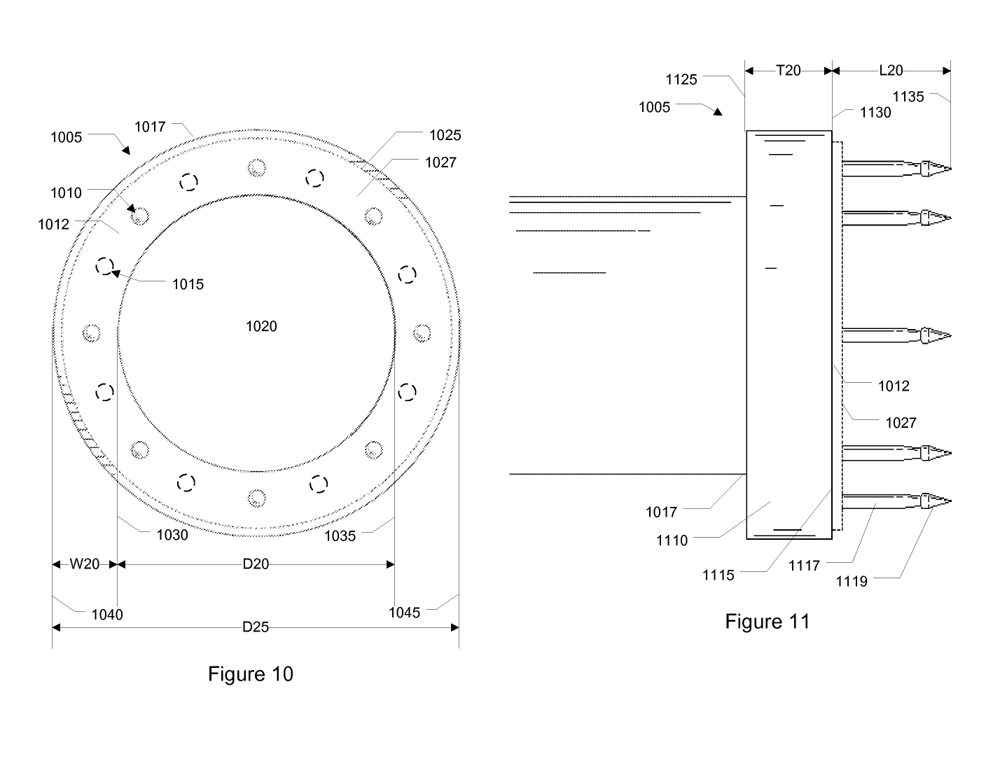

FIG. 10 shows a front view of a coupler ring 1005 with pins 1010 on a first side 1012, pin openings 1015 (shown as dotted lines) on a second side 1017, and a vessel opening 1020 through which a tube 1025 is passed through. An everted portion 1027 of the tube is impaled on the pins.

Typically, the pins are evenly distributed about the vessel opening. For example, an angle between the pins is given by 360 degrees divided by the total number of pins (e.g., eight pins, the angle is 45 degrees; six pins, the angle is 60 degrees).

Similarly, the pin openings which are positioned between the pins are typically evenly distributed about the vessel opening. For example, an angle between the pin openings is given by 360 degrees divided by the total number of pin openings (e.g., eight pin openings, the angle is 45 degrees; six pin openings, the angle is 60 degrees). In other embodiments, the pins, pin openings, or both are not evenly distributed about the vessel opening.

An even distribution of the pin and pin openings about the vessel opening allows, for example, an even distribution of frictional force about the vessel opening of the coupler ring when two coupler rings are joined. For example, first and second coupler rings are joined when the pins of the first coupler ring are inserted into the pin openings of the second coupler ring. And, the pins of the second coupler ring are inserted into the pin openings of the first coupler ring. The two coupler rings are held together, at least in part, by the frictional force between the surface of the pins and the inner surface of the pin openings.

In a specific embodiment, the frictional force is augmented by processing the pins, pin openings, or both to make a surface that is rougher than the original starting surface. For example, one or more pins may have a textured or knurled surface via bead blasting or machining. This can increase the frictional force between the surface of the pins and the inner surface of the pin openings to help prevent the two rings from accidentally separating.

In another embodiment, the frictional force is augmented by processing the pins, pin openings, or both to make a surface that is smoother than the original starting surface. For example, friction increases between two extremely smooth surfaces because of increased attractive electrostatic forces between their atoms.

Similarly, one or more pins, pin openings, or both may be coated with an adhesive (e.g., pressure sensitive adhesive and glue) to help prevent the two rings from accidentally separating.

A distance D20 (i.e., vessel opening diameter) is between lines 1030 and 1035. A distance D25 (i.e., coupler ring diameter) is between lines 1040 and 1045. A distance W20 (i.e., coupler ring width) is between lines 1030 and 1040.

The vessel opening diameter is generally similar to the outer diameter of the tube to which the coupler ring is attached. This allows the tube to be passed through the vessel opening without having to squeeze the tube in order to pass the tube through the vessel opening. The tube can also be everted or stretched over the pins without the tube being overly stretched such that it tears.

The tube may be made of a material that is stretchable like a balloon. Thus, in a specific embodiment, the vessel opening diameter is larger than the outer diameter of the tube. The tube's stretchable properties allow the tube to be radially stretched towards and over the pins without tearing.

Table B below shows dimensions-vessel opening diameter D20, coupler ring diameter D25, coupler ring width W20, coupler ring thickness T20 (FIG. 11), and pin length L20 (FIG. 11)—for various implementations of the invention, and also a range of dimensions. However, it should be noted that these dimensions may vary greatly depending upon the application.

TABLE B

| | Coupler ring | | | | |
|---|---|---|---|---|---|
| | vessel | | | Coupler ring | |
| | opening | Coupler ring | | base | |
| | diameter | diameter | Coupler ring | thickness | Pin length |
| | (D20, | (D25, | width (W20, | (T20, | (L20, |
| Implementation | millimeters) | millimeters) | millimeters) | millimeters) | millimeters) |
| First | 0.94 | 2.29 | 0.68 | 0.61 | 0.81 |
| Second | 1.45 | 2.79 | 0.67 | 0.71 | 0.81 |
| Third | 1.96 | 3.40 | 0.72 | 0.71 | 1.11 |
| Fourth | 2.41 | 3.89 | 0.74 | 0.89 | 1.11 |
| Fifth | 3 | 4 | 1 | 1 | 1.5 |
| Sixth | 3.5 | 4.2 | 1.1 | 1 | 1.5 |
| Seventh | 4 | 4.2 | 1.1 | 1.1 | 1.6 |
| Eighth | 4.5 | 4.9 | 1.2 | 1.2 | 1.6 |
| Ninth | 5 | 5.5 | 1.1 | 1.3 | 1.7 |
| Tenth | 5.5 | 6.5 | 1.5 | 1.5 | 1.7 |
| Eleventh | 6 | 7 | 2 | 2.5 | 2 |
| Twelfth | 7 | 8 | 2.5 | 2.5 | 2 |
| Thirteenth | 8 | 9.5 | 2.7 | 2.6 | 2.1 |
| Fourteenth | 9 | 10 | 3 | 2.9 | 2.5 |
| Fifteenth | 10 | 10.9 | 3.2 | 2.9 | 2.5 |
| Sixteenth | 11 | 12 | 3.5 | 2.9 | 2.5 |
| Seventeenth | 12 | 13 | 4 | 3 | 3 |
| Eighteenth | 0.5-14 | 1-18 | 0.5-5 | 0.5-4 | 0.6-5 |

The vessel opening of the coupler ring can have any shape. Although FIG. 10 shows the vessel opening with a circular shape, other embodiments include a vessel opening with, for example, an oval shape, elliptical shape, rectangular shape, square shape, octagonal shape, or triangular shape.

For example, in a specific embodiment, the vessel opening of the coupler ring has an oval or elliptical shape with a major and minor axis. Table C below shows dimensions—vessel opening minor axis, vessel opening major axis, coupler ring diameter D25, coupler ring width W20, coupler ring base thickness T20 (FIG. 11), and pin length L20 (FIG. 11)—for various implementations of the invention, and also a range of dimensions in millimeters. However, it should be noted that these dimensions may vary greatly depending upon the application.

TABLE C

| | Coupler | Coupler | Coupler | | Coupler | |
|---|---|---|---|---|---|---|
| | ring vessel | ring vessel | ring | Coupler | ring base | |
| | opening | opening | diameter | ring width | thickness | Pin length |
| Implementation | minor axis | major axis | (D25) | (W20) | (T20) | (L20) |
| First | 2.92 | 3.18 | 4.60 | 0.78 | 0.96 | 1.11 |
| Second | 3.33 | 3.84 | 5.36 | 0.88 | 0.91 | 1.40 |
| Third | 3.60 | 4.34 | 6.04 | 1.04 | 0.96 | 1.40 |
| Fourth | 1-10 | 1.2-15 | 2-20 | 0.5-5 | 0.5-5 | 0.5-5 |

FIG. 11 shows a side view of coupler ring 1005 attached to the tube. The coupler ring includes a base 1110 with a front surface 1115 from which the pins project outwards.

The vessel extender is typically assembled by the manufacturer by passing the tube through the coupler ring and attaching the tube to the coupler ring. That is, the tube is passed through the vessel opening from second side 1017 to first side 1012 where everted portion 1027 of the tube is impaled on the pins.

In another embodiment, the tube does not pass through to first side 1012. The tube passes from the second side and towards the first side, but the tube terminates before reaching the first side. In this specific embodiment, the tube is not connected to the coupler ring by impaling the tube onto the pins. Instead, an adhesive (e.g., fibrin glue and epoxy) between the tube and the vessel opening of the coupler ring is used to secure the tube to the coupler ring.

An adhesive may also be used in the embodiment where the tube is impaled onto the pins. For example, an adhesive may be placed between the front surface of the coupler ring and the everted portion of the tube. This can help to ensure that the coupler ring does not accidentally separate from the tube. This can also help to ensure that the openings created on the everted portion of the tube when the pins pierce the tube do not result in tears which propagate from those openings

The pins include a shaft portion 1117 and a barb portion 1119 which helps to prevent the pin from pulling out of a pin opening. In a specific embodiment, the pin further includes one or more sharp projections extending backward (i.e., extending towards the base) which help to prevent the pin from being easily extracted from the pin opening.

In a specific implementation, the diameter of the shaft portion is about 0.28 millimeters, but may range from about 0.1 millimeter to about 2 millimeters, including less than 0.1 millimeter and greater than 2 millimeters. Typically, the pins (e.g., shaft portion and barb portion) have circular cross sections, but can have a cross section having any shape such as a square, rectangle, oval, triangle, or combinations of these.

The pins are positioned such that they are perpendicular to the front surface. However, in other embodiments, one or more pins are positioned such that they are at an angle to the front surface.

A distance T20 (i.e., coupler ring base thickness) is between lines 1125 and 1130. A distance L20 (i.e., pin length) is between lines 1130 and 1135. In a specific embodiment, the thickness of the base of the coupler ring remains constant. However, in other embodiments, the thickness varies.

The base is typically made of plastic (e.g., acrylics, polyesters, silicones, polyurethanes, halogenated plastics, polystyrene, and polyvinyl chloride), nylon, or fiberglass. But the base may also be made of other materials or combinations of other materials such as metal (e.g., steel, aluminum, and titanium), ceramics, composites (e.g., carbon fiber), or rubber.

The pins are typically made of metal such as stainless steel (e.g., 316L stainless steel), nickel, nickel titanium, titanium, zirconium, gold, niobium, magnesium, or platinum, but may also be made of plastic (e.g., acrylics, polyesters, silicones, polyurethanes, halogenated plastics, polystyrene, and polyvinyl chloride), nylon, fiberglass, ceramics, or composites (e.g., carbon fiber).

In a specific implementation, at least two of the vessel extender elements (i.e., coupler ring base, pins, and tube) are formed as a single or one-piece or integrated unit. For example, the coupler ring base, pin, and tube may be formed as a one-piece unit via injection molding or reaction injection molding. In this specific implementation, two or more vessel extender elements are not formed as separate independent elements that are then put together. Rather, in this specific implementation, two or more vessel extender elements are manufactured as integrated units. This can help to reduce the manufacturing costs by eliminating the step of putting together separate independent elements.

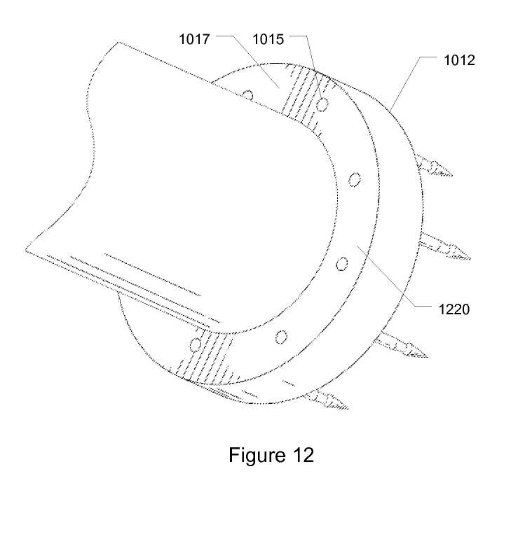

FIG. 12 shows a perspective view of second side 1017 of the vessel extender. In a specific embodiment, pin openings 1015 pass from second side 1017 to first side 1012 where the pin openings are then covered by the everted portion of the tube. That is, in a specific embodiment, the length of the pin openings is the same as the thickness of the base. In other embodiments, the length of the pin openings is different from the thickness of the base.

Generally, the length of the pin openings is such that when the coupler rings are joined, the pins do not protrude past a back surface 1220 of the base on second side 1017. That is, the pins of the first coupler ring of the vessel extender when inserted into the pin openings of the third coupler ring attached to the body vessel typically do not protrude past the back surface of the base of the third coupler ring. This is generally desirable because the ends of the pins are typically sharp points. If these sharp points protrude past the back surface then these sharp points may cause contact damage to the surrounding tissue.

The pin generally has a length (L20, FIG. 11) that is sufficient to pierce through the everted portion of the tube, the everted portion of the body vessel impaled onto the third coupler ring, and enter the pin opening of the third coupler ring, but not extend past the back surface of the third coupler ring.

Thus, the length of a pin opening is typically a function of at least the length of a pin, the thickness of the everted portion of the tube, and the thickness of the everted portion of the body vessel. The length of the pin opening is represented by the following equation:

length of pin opening≧L20−(T30+T40) (2)

where L20 is the pin length (FIG. 11), T30 is the thickness of the everted portion of the tube, and T40 is the thickness of the body vessel.

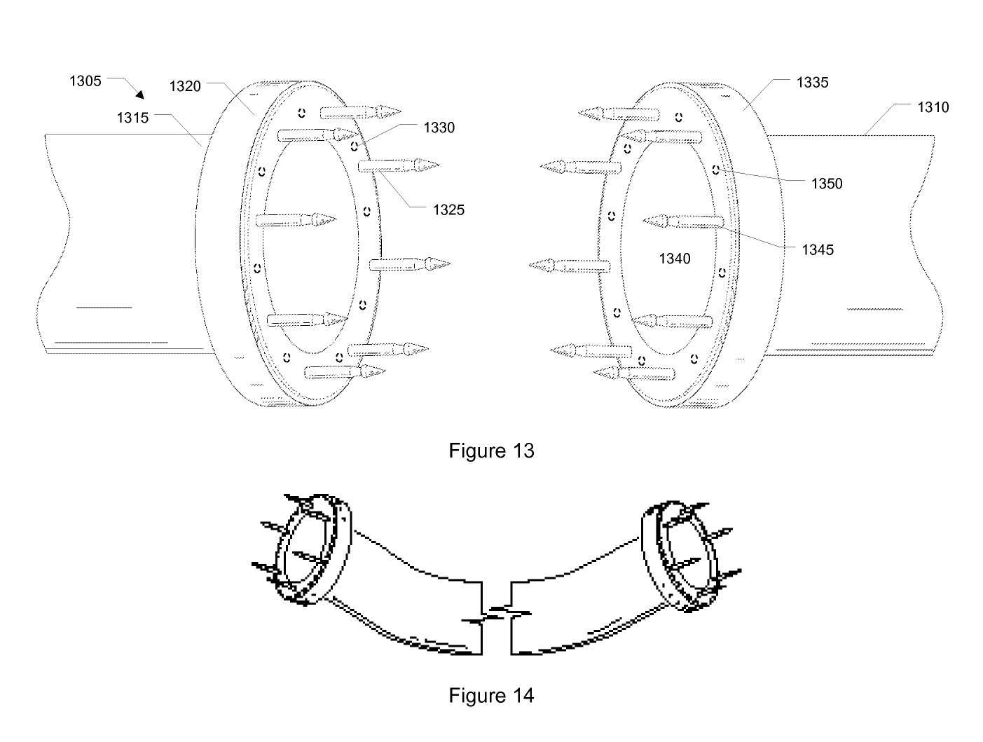

FIG. 13 shows a perspective view of a vessel extender 1305 in the process of being joined to a body vessel 1310. An end 1315 of the vessel extender includes a first coupler ring 1320 that includes pins 1325 and pin openings 1330. The second coupler ring of the vessel extender has been omitted from this view for sake of clarity. A third coupler ring 1335 has been attached to the body vessel by passing the body vessel through a vessel opening 1340 of the third coupler ring, everting the body vessel over pins 1345, and impaling the body vessel onto the pins.

End 1315 of the vessel extender is joined to the body vessel by squeezing the first and third coupler rings together such that pins 1325 of the first coupler ring mate with pin openings 1350 of the third coupler ring, and pins 1345 of the third coupler ring mate with pin openings 1330 of the first coupler ring.

In a specific embodiment, the coupler ring includes eight pins as shown in FIG. 13. In another embodiment, the coupler ring includes six pins as shown in FIG. 14 (a perspective view of a vessel extender with coupler rings having six pins). The number of pins on a coupler ring ranges from about four pins to about twelve pins, including, for example, five, six, seven, eight, nine, ten, eleven pins, more than twelve pins, or less than four pins. Generally, the number of pins is proportional to the diameter of the vessel opening on the coupler ring. That is, the greater the diameter of the vessel opening, the greater the number of pins on the coupler ring.

In a specific embodiment, the coupler ring includes eight pin openings as shown in FIG. 13. In another embodiment, the coupler ring includes six pin openings. The number of pin openings on a coupler ring ranges from about four pin openings to about twelve pin openings, including, for example, five, six, seven, eight, nine, ten, eleven pin openings, more than twelve pin openings, or less than four pin openings. Generally, the number of pin openings is proportional to the vessel opening diameter of the coupler ring. That is, the greater the diameter of the vessel opening, the greater the number of pin openings on the coupler ring.

Typically, the number of pins on the first coupler ring is the same as the number of pin openings on the third coupler ring (or the coupler ring that the first coupler ring will join with). For example, if the first coupler ring has eight pins then the third coupler ring will have eight pin openings in order to accept the eight pins on the first coupler ring.

However, in another embodiment, the number of pins on the first coupler ring is different from the number of pin openings on the third coupler ring. For example, the number of pins on the first coupler ring may be fewer or more than the number pin openings on the third coupler ring.

In various embodiments, a coupler ring includes pins and no pin openings, pin openings and no pins, or no pins and no pin openings. For example, in a first embodiment, a first coupler ring includes pins and no pin openings. A second coupler includes pin openings and no pins. The first and second coupler rings can still be joined because the pins of the first coupler ring can mate with the pin openings of the second coupler ring. Such a design can help to reduce manufacturing costs because pins are attached only to one coupler ring instead of two coupler rings.

In a second embodiment, a first coupler ring and a second coupler ring do not have any pins or pin openings. Instead, the first coupler ring and second coupler ring are joined using an adhesive (e.g., epoxy and fibrin glue) placed between the first and second coupler rings.

In a third embodiment, a first coupler ring includes pins and a second coupler ring does not have any pin openings. Instead the pins on the first coupler ring pierce through the material of the second coupler ring. The first and second coupler rings are then held together, at least in part, by the frictional force between the pins and openings created in the material by the pins piercing the coupler ring material.

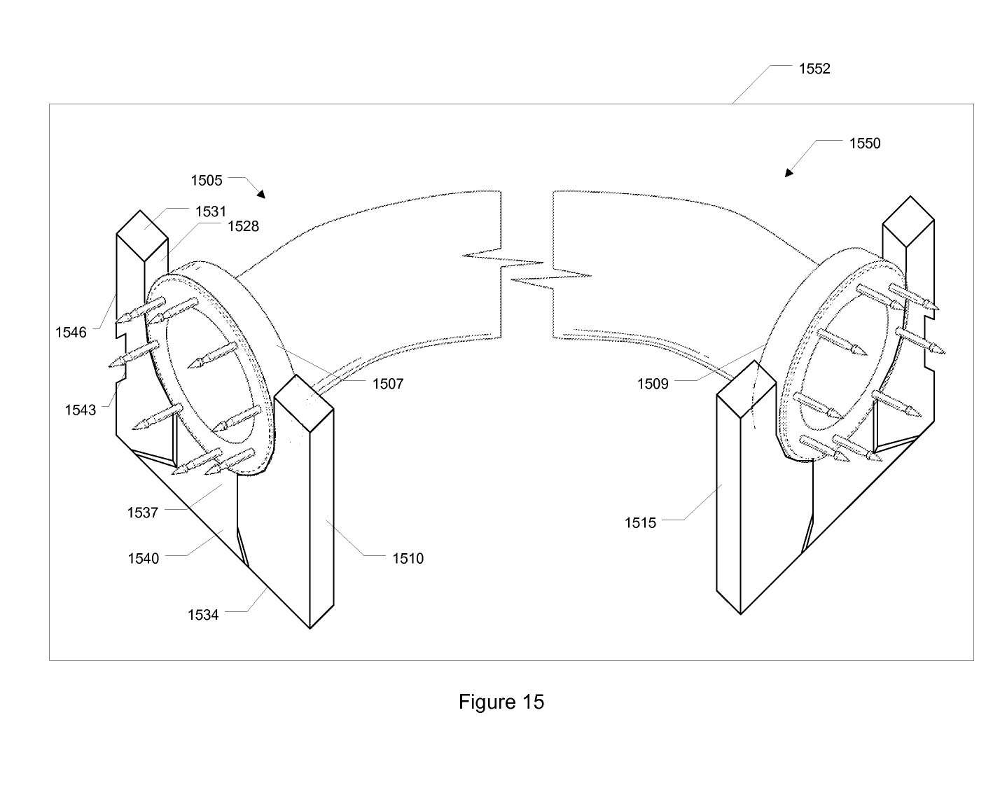

FIG. 15 shows a perspective view of a vessel extender 1505 with first and second coupler rings 1507 and 1509 that are held by first and second jaw assemblies 1510 and 1515, respectively.

A jaw assembly, such as the first jaw assembly includes a U-shaped slot 1528 which at least partially surrounds and holds the first coupler ring. The U-shaped slot extends from a top side 1531 of the jaw assembly, towards a bottom side 1534 of the jaw assembly. The bottom of the U-shaped slot is connected to a straight slot 1537, which is connected to an angled slot 1540. A cavity 1543 is on a side edge 1546 of the jaw assembly.

Typically, the frictional force between the jaw assembly and the coupler ring prevents the jaw assembly from accidentally sliding off of the coupler ring. For example, typically the base of the coupler ring is made of a flexible or semiflexible material such as plastic. The base can be slightly squeezed or compressed so that it resiliently deforms such that it can squeeze into the U-shaped slot.

In a specific embodiment, an alignment mechanism is included with the coupler ring, jaw assembly, or both to orient and position the coupler ring in the jaw assembly. The alignment mechanism can help to ensure that when joining two coupler rings that the pins of one coupler ring align with the pin openings of the other coupler ring.

The alignment mechanism may be a tab that extends from a side surface of the coupler ring and into straight slot 1537. As another example, a portion of the side surface of the coupler ring includes a tongue, rib, or ridge which slides into a grove on the jaw assembly. As another example, the coupler ring includes a first indicator (e.g., first arrow) and the jaw assembly includes a second indicator (e.g., second arrow). The user orients the coupler ring in the jaw assembly by matching the first and second arrows. As a further example, at least a portion of the side surface of the coupler ring has a flat edge which slides along the U-shaped slot.

In a specific embodiment, the jaw assemblies and vessel extender are packaged as a single unit 1550 as shown in the example in FIG. 15. That is, the jaw assemblies and vessel extender are packaged in a package 1552 that is sealed using, for example, an adhesive, heat (e.g., heat sealing) or a vacuum (e.g., vacuum sealing). The sealing helps to prevent bacteria and debris from entering the inside of the package and maintains a sterile environment for the contents.

The package may include information that identifies the size of the vessel extender (e.g., length and diameter), the date that the vessel extender was packaged, and the expiration date of the vessel extender. The information may also include warnings and instructions for storage and use (e.g., “keep away from direct sunlight,” “keep cool,” “fragile,” and “tear here”). Such information, warnings, and instructions may be printed on the package, printed on one or more labels attached to the package, or printed on an instruction manual that accompanies the package.

In a specific implementation, the instructions describe the process for rehydrating the vessel extender where the tube of the vessel extender includes freeze-dried tissue, such as freeze-dried human tissue. The instructions include soaking the vessel extender in a bath of warm saline solution for certain time period (e.g., 10 minutes, 20 minutes, 30 minutes, and 40 minutes) and the threshold time period (e.g., 1 hour, 2 hours, 3 hours, and 4 hours) within which the vessel extender should be used after rehydration.

In this specific implementation, the coupler rings are not attached to the tube. Instead, the coupler rings are included in a separate package that accompanies the tube. This is because the freeze-drying of the tissue typically causes the tissue to shrink. If the coupler rings are attached to the tube during the freeze-drying process the shrinking of the tube may result in the tube tearing away from the coupler rings.

However, in another implementation, the coupler rings are attached to the freeze-dried tube. This saves the doctor the step of having to attach the coupler rings to the rehydrated tube. For example, the coupler rings may be made of a material (e.g., shape-memory polymer) that can similarly shrink with the tube during the freeze-dry process or another process.

Examples of packages include bags (e.g., foil-lined pouches and blister packs) or rigid containers (e.g., box). A bag is typically less expensive to manufacture than a rigid container, but a rigid container offers greater protection than a bag during shipping and storage.

Thus, the vessel extender is typically designed as an off-the-shelf item. The doctor can open the package at the time of actual use or near the time of actual use so as to not contaminate the vessel extender. The vessel extender can be used in cases where a portion of a blood vessel is needed. For example, in a typical bypass operation, the doctor removes blood vessels (e.g., veins) from the donor site (e.g., patient's leg) and uses the vessels to reroute blood around blocked arteries at the recipient site (e.g., patient's chest). However, suitable veins can be hard to find, especially in patients who have had previous bypass surgeries, are obese, or are diabetic. The additional incision at the donor site to harvest vessels also creates the potential for additional problems such as infections. With the vessel extender, such problems can be avoided.

The jaw assemblies, unlike the vessel extender (i.e., tube and coupler rings), are typically not designed to remain in situ in the body. Instead, the jaw assemblies aid the user (e.g., doctor) in performing the anastomosis. That is, the jaw assembly makes the vessel extender easier to handle. For example, the coupler rings are typically very small, i.e., having diameters from about 1 millimeter to about 14 millimeters. With the jaw assemblies, the doctor can use tweezers or forceps to grip the jaw assembly and insert the jaw assembly into an anastomotic coupler assembly tool. Cavity 1543 may be designed to snap into a protrusion on the anastomotic coupler assembly tool. The doctor can then use the anastomotic coupler assembly tool to connect the body vessel ends. By gripping the jaw assembly instead of the coupler ring or the tube, there is less chance that the coupler ring, tube, or both will become damaged (e.g., crushed by the tweezers).

Another benefit of the jaw assemblies is that they are disposable after use. That is, the user can remove the jaw assemblies from the anastomotic coupler assembly tool and throwaway the jaw assemblies. This helps to prevent contamination of the tool during the surgical procedure.

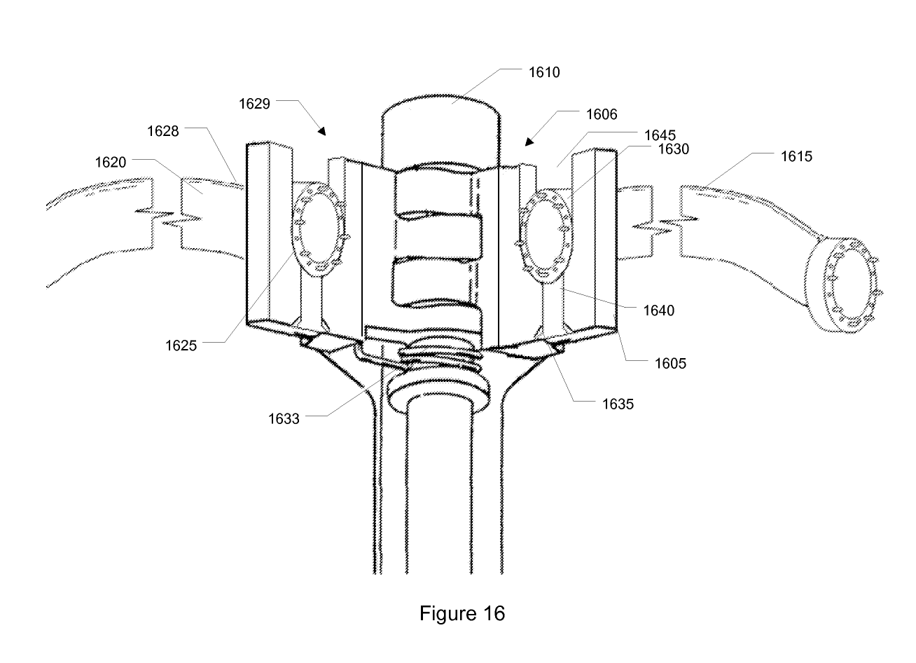

FIG. 16 shows a first jaw assembly 1605 attached to a first side 1606 of a specific implementation of an anastomotic coupler assembly tool 1610. The figure shows a vessel extender 1615 that is in the process of being attached to a body vessel 1620 (e.g., artery and vein) with the aid of the anastomotic coupler assembly tool. Another assembly tool is discussed in U.S. provisional patent application 61/093,185, filed Aug. 29, 2008, which is incorporated by reference.

A third coupler ring 1625 has been connected to an end 1628 of the body vessel and is being held in a second side 1629 of the tool. The first jaw assembly is holding a first coupler ring 1630 of the vessel extender.

This specific implementation of the anastomotic coupler assembly tool includes a hinge 1633 about which the first and third coupler rings will rotate towards each other, thus becoming joined. As the first and third coupler rings rotate towards each other, a push rod on the tool enters an angled slot 1635, passes through a straight slot 1640, enters into U-shaped slot 1645, and pushes the first coupler ring. As the first and third coupler rings continue to rotate towards each other, the push rod continues to push on the first coupler ring which in turn causes the first coupler ring to slide out of the U-shaped slot.

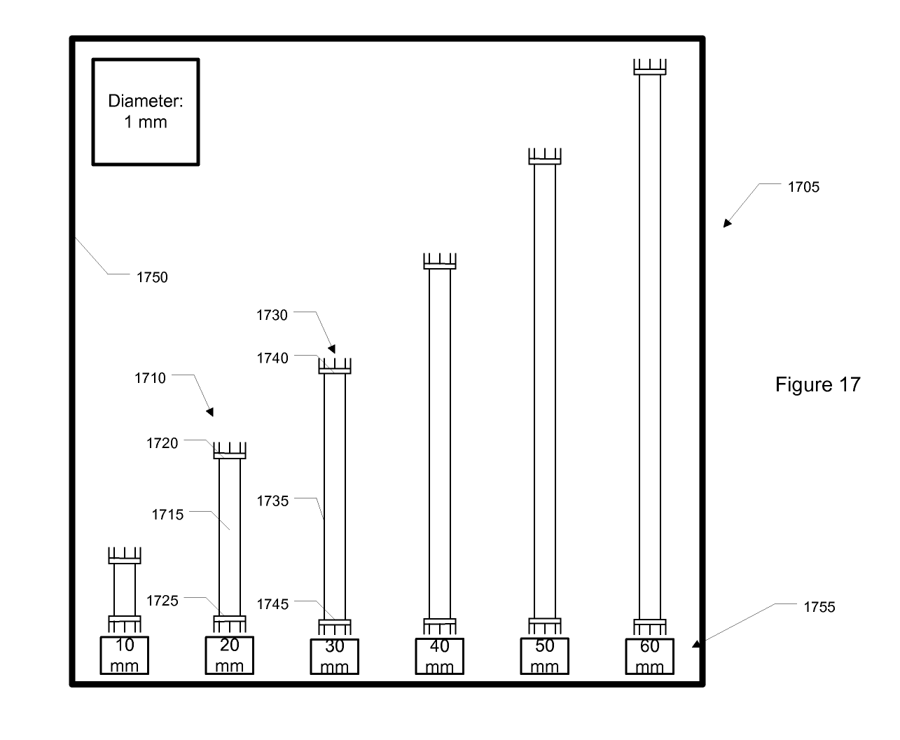

FIG. 17 shows a first implementation of a packaging option for the vessel extenders. In a specific implementation, two or more vessel extenders are provided as a vessel extender kit 1705. In this specific implementation, kit 1705 includes an assortment of vessel extenders having the same diameters (e.g., inner diameters and outer diameters), but having different lengths (e.g., 10 millimeters, 20 millimeters, 30 millimeters, 40 millimeters, 50 millimeters, and 60 millimeters).

For example, a first vessel extender 1710 includes a first tube 1715 connected to first and second coupler rings 1720 and 1725. The first tube has a first diameter and a first length. A second vessel extender 1730 includes a second tube 1735 connected to third and fourth coupler rings 1740 and 1745. The second tube has a second diameter and a second length. The first diameter is the same as the second diameter. The first length is different from the second length. As shown in the example in FIG. 17, the second length is greater than the first length.

The vessel extenders are placed into a container 1750. The container includes a base member, a recloseable lid, and a tray that fits into the base member. The recloseable lid may be made of a transparent material such as clear plastic so that the vessel extenders in the kit are visible to a user without the user having to open the container. In an embodiment, the recloseable lid and base member are connected with a hinge that allows the recloseable lid to swing open and swing close (e.g., clamshell container).

The tray includes cavities to hold each vessel extender. The cavities may be coated in an antibacterial coating to help prevent bacterial growth. Clips attached to the tray and adjacent to the cavities may also be included to secure a vessel extender within a cavity so that the vessel extender does not shift during transportation.

A vessel extender may also be sealed in a package within the container to provide a sterile environment for the vessel extender. This helps to prevent the vessel extenders from becoming contaminated each time the container is opened and closed.

In various embodiments, the tray is made of plastic, foam, or polypropylene. Labels 1755 adjacent to the cavities identify the vessel extenders (e.g., identify the length of the vessel extender). Some examples of labels include stickers and tags. The labels may include text (e.g., 10 millimeters, 20 millimeters, 30 millimeters, 40 millimeters, 50 millimeters, and 60 millimeters). In other embodiments, the labels are color, number, symbol, or letter codes which identify the vessel extender. A color, number, symbol, or letter legend may be provided so that users can identify the vessel extender.

As shown in the example of FIG. 17, the kit includes six vessel extenders. However, it should be appreciated that a kit may include any number of vessel extenders, such as two, three, four, five, six, seven, eight, nine, ten vessel extenders, or more than ten vessel extenders.

Furthermore, some embodiments of the kit include one or more vessel extenders with the jaw assemblies attached to the coupler rings. However, the jaw assemblies are optional and are not included in some embodiments of the kit.

The kit may further include an expiration date (e.g., “month-day-year”) to indicate a date after which the vessel extenders in the kit should no longer be used. For example, an exterior surface of the container may include a label onto which the expiration date is printed.

In a specific embodiment, the kit includes an environmental event indicator which indicates whether the kit and thus the vessel extenders have been subjected to adverse or undesirable environmental conditions. The environmental event indicator is typically attached to a surface (e.g., exterior surface and interior surface) of the container so that it is visible to a user without the user having to open the container. Environmental factors which may degrade the performance of the vessel extenders include, for example, temperature, humidity, and radiation.

As an example, various embodiments of the vessel extender include materials (e.g., cells and freeze-dried tissue) which must be maintained at a certain temperature or a range of temperatures. If, for example, the ambient temperature exceeds a threshold hold temperature or falls below a threshold hold temperature for a threshold period of time this may damage the vessel extender. For example, the compliance of the tube of the vessel extender may decrease if the ambient temperate exceeds a threshold temperature for a threshold period of time. This is undesirable because the tube will not be able to properly expand, stretch, dilate, or contract. If a doctor provides a patient with this compromised vessel extender then the patient's safety will be at risk.

Thus, in a specific embodiment, the kit (or package containing a vessel extender) includes a thermal event indicator such as an irreversible low temperature indicator, an irreversible high temperature indicator, or both. The irreversible low temperature indictor includes a first visual indicator such as a first dye which changes from a first color to a second color when the ambient temperature has fallen below a first threshold temperature for a first threshold period of time.

Similarly, the irreversible high temperature indicator includes a second visual indicator such as a second dye which changes from a third color to a fourth color when the ambient temperature has risen above a second threshold temperature for a second threshold period of time.

The color change (i.e., first color to second color and third color to fourth color) is permanent or irreversible. Thus, if the vessel extenders in the kit are exposed to stressful temperature conditions and then returned less stressful temperature conditions, the user will still be able to see the colors (i.e., second color and fourth color) and know that vessel extenders are no longer reliable and should not be placed in a patient.

In another embodiment, the kit includes a portable, battery-operated heating system, cooling system, humidifier, dehumidifier, or combinations of these to help maintain the temperature and humidity levels in the kit.

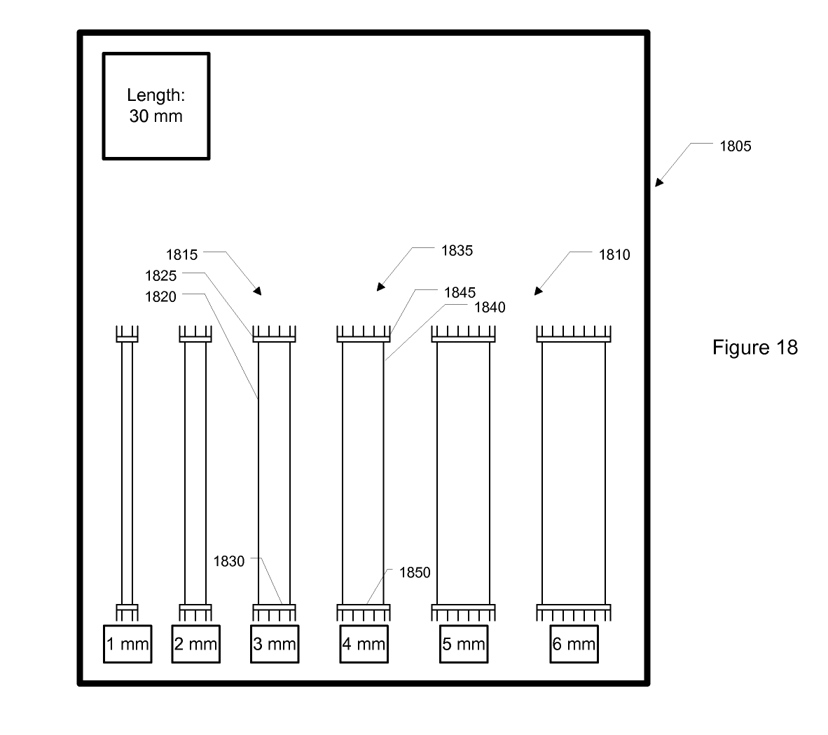

FIG. 18 shows a second implementation of a packaging option for the vessel extenders. In this specific implementation, a kit 1805 includes an assortment of vessel extenders 1810 having the same length (e.g., tube length), but having different diameters such as different inner diameters, outer diameters, or both.

In a specific embodiment, a first vessel extender 1815 includes a first tube 1820 connected to first and second coupler rings 1825 and 1830. The first tube has a first diameter and a first length. A second vessel extender 1835 includes a second tube 1840 connected to third and fourth coupler rings 1845 and 1850. The second tube has a second diameter and a second length. The first diameter is different from the second diameter. The first length is the same as the second length. As shown in the example in FIG. 18, the second diameter is greater than the first diameter.

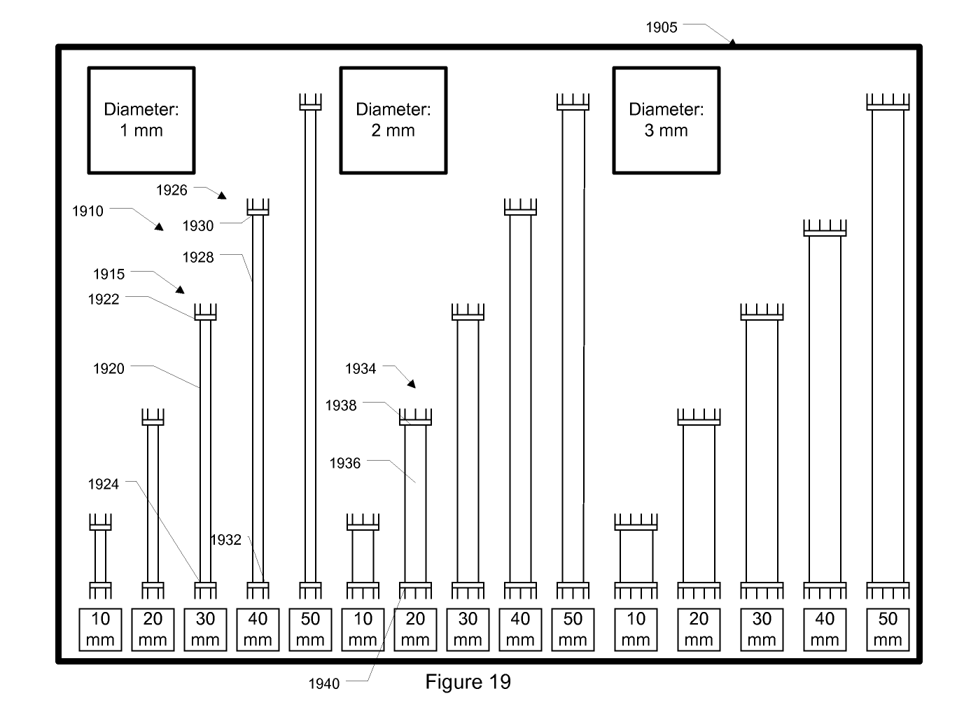

FIG. 19 shows a third implementation of a packaging option for the vessel extenders. In this specific implementation, a kit 1905 includes an assortment of vessel extenders 1910 with varying diameters (e.g., 1 millimeter, 2 millimeters, and 3 millimeters) and varying lengths (e.g., 10 millimeters, 20 millimeters, 30 millimeters, 40 millimeters, and 50 millimeters).

In a specific embodiment, a first vessel extender 1915 includes a first tube 1920 connected to first and second coupler rings 1922 and 1924. The first tube has a first diameter and a first length.

A second vessel extender 1926 includes a second tube 1928 connected to third and fourth coupler rings 1930 and 1932. The second tube has a second diameter and a second length.

A third vessel extender 1934 includes a third tube 1936 connected to fifth and sixth coupler rings 1938 and 1940. The third tube has a third diameter and a third length.

The first diameter is the same as the second diameter. The first diameter is different from the third diameter. The first length is different from the second length and the third length.

Other embodiments of the kit include two or more vessel extenders having the same lengths, same diameters, or both. Or two or more vessel extenders having different lengths, different diameters, or both.

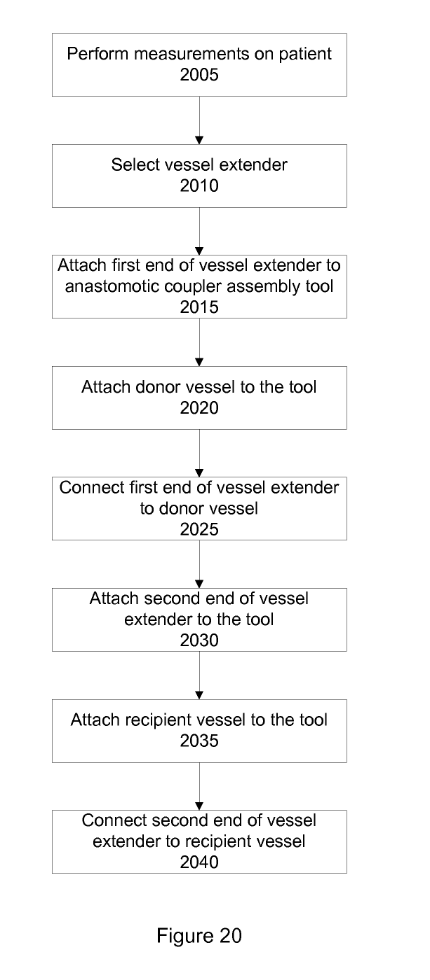

FIG. 20 shows a flow diagram representative of a user using a vessel extender. The user may be a person (e.g., doctor), robot, or computer. In a specific implementation, the vessel extender is implanted in a patient using an endoscopic instrument or a robotic arm having a robotic interface. The robotic interface allows a doctor to control the endoscopic instrument or robotic arm from a remote location. For example, the doctor in New York City can use the endoscopic instrument or robotic arm to perform a remote procedure (e.g., remote microvascular anastomosis) on a patient who is located in Barrows, Ak. The robotic interface may have a haptic interface which provides feedback to the doctor or may not have a haptic interface. When a haptic interface is not available, the readings provided by the endoscopic instrument or robotic arm may give the doctor an indication of the status of the procedure.

In a step 2005, the user performs a series of measurements of the patient in order to determine what size (e.g., diameter and length) vessel extender should be used.

For example, a first measurement includes measuring a path length between the two body vessel ends (i.e., between first and second blood vessel ends; or between donor and recipient blood vessel ends) that are to be connected. Factors that may influence the path length include the distance between the donor and recipient body vessel ends when the donor and recipient vessels are in situ in the body, the thickness of the coupler rings on the donor and recipient blood vessel ends, whether the vessel extender must be routed around any obstructions between the donor and recipient blood vessel ends, and any margins of length that the user desires to account for tools in the surgical site such as vascular clamps that may be placed on the donor and recipient blood vessels.

A second measurement includes measuring a first diameter of the donor blood vessel end. A third measurement includes measuring a second diameter of the recipient blood vessel end.

Any technique may be used to make these measurements. For example, to measure the diameters, the user may use a handheld body vessel diameter gauge or a tape measure. To measure the path length, the user may use the tape measure to measure the distance between the two body vessel ends. The user may be aided by a microscope, magnifying glass, or an ultrasound machine or an x-ray machine with image analysis software.

In a step 2010, the user selects the vessel extender. For example, the vessel extender may be selected from a vessel extender kit as discussed above. The selected vessel extender typically has a length (e.g., tube length plus thickness of coupler rings) that is similar to or at least as long as the path length. That is, the selected vessel extender will typically not have a length that is less than the path length. The selected vessel extender also typically has a diameter (e.g., inner diameter and outer diameter) that is similar to the diameter of the vessels ends.

In a step 2015, the user attaches the first end of the vessel extender to the first side of the anastomotic coupler assembly tool. For example, the user may snap the first jaw assembly, which holds the first coupler ring of the vessel extender, into the first side of the tool.

In a step 2020, the user attaches the donor blood vessel end to the tool. That is, after the user attaches the third coupler ring to the donor blood vessel end, the user attaches the third coupler ring to the second side of the tool.

In a step 2025, the user uses the tool to connect the first end of the vessel extender to the donor blood vessel end. For example, the user uses the tool to bring the first and second sides of the tool together, thereby joining the third coupler ring on the donor blood vessel with the first coupler ring of the vessel extender.

When the anastomosis of the first end of the vessel extender to the donor blood vessel end is complete, the user can proceed to join the second end of the vessel extender to the recipient blood vessel end. That is, in a step 2030, the user attaches the second end of the vessel extender to the tool. For example, the user may snap the second jaw assembly, which holds the second coupler ring of the vessel extender, into the first side of the tool

In a step 2035, the user attaches the recipient blood vessel end to the tool. That is, after the user attaches the fourth coupler ring to the recipient blood vessel end, the user attaches the fourth coupler ring to the second side of the tool.

In a step 2040, the user uses the tool to connect the second end of the vessel extender to the recipient blood vessel end by, for example, bringing the first and second side of the tool together and joining the fourth coupler ring on the recipient blood vessel with the second coupler ring of the vessel extender.

FIG. 20 describes a flow for an end-to-end anastomosis. The user can follow a similar procedure for a side-to-end anastomosis.

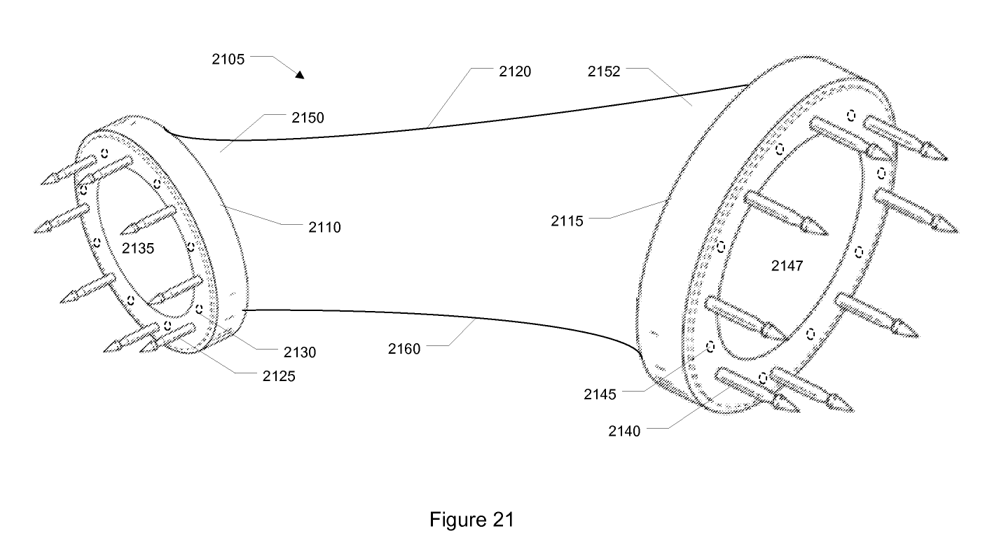

FIG. 21 shows a side view of a fifth embodiment of a vessel extender 2105. This embodiment of the vessel extender may be referred to as a “vessel right sizer.” The vessel right sizer includes a first coupler ring 2110, a second coupler ring 2115, and a tube 2120 between the first and second coupler rings. The first coupler ring includes a first set of pins 2125, a first set of pin openings 2130, and a first vessel opening 2135. The second coupler ring includes a second set of pins 2140, a second set of pin openings 2145, and a second vessel opening 2147. The tube is connected between the first and second vessel openings.

In this specific embodiment, the diameters of the first and second vessel openings are different. As shown in the example of FIG. 21, the diameter of the second vessel opening is greater than the diameter of the first vessel opening. The diameter of the second vessel opening may be 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, or 950 percent greater than the diameter of the first vessel opening. Depending upon the application, the diameter of the second vessel opening may be less than 5 percent or more than 950 percent greater than the diameter of the first vessel opening.

A ratio of the diameter of the first vessel opening (or the diameter of the smaller vessel opening) to the diameter of the second vessel opening (or the diameter of the larger vessel opening) is typically in the range of about 1:1.1 to about 1:10. Some examples of the ratio include 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:4, 1:4.5, 1:5, 1:5.5, 1:6, 1:6.5, 1:7, 1:7.5, 1:8, 1:8.5, 1:9, 1:9.5, or 1:9.9.

The different diameters at the first and second ends of the tube and the first and second vessel openings allow the vessel right sizer to join two body vessels (e.g., first and second blood vessels or donor and recipient blood vessels) that have different inner diameters, outer diameters, or both.

For example, in some cases the diameter of a recipient blood vessel will be different from the diameter of a donor blood vessel. That is, the diameter of the recipient blood vessel will be smaller or larger than the diameter of the donor blood vessel. When selecting a coupler ring to attach to the blood vessels it is desirable to select a coupler ring with a vessel opening diameter that is similar to the diameter of the blood vessel.

If the vessel opening diameter of the coupler ring is much smaller than the diameter of the blood vessel then the user will have to squeeze the blood vessel in order to pass the blood vessel through the vessel opening of the coupler ring. The squeezing of the blood vessel may result in undesirable folds or creases in the blood vessel which may damage the blood vessel. Conversely, if the vessel opening diameter of the coupler ring is much greater than the diameter of the blood vessel then the user may overly stretch the blood vessel while impaling the blood vessel onto the pins. This can lead to the blood vessel tearing.

Thus, in cases where the diameter of the donor and recipient blood vessels are different, the vessel opening diameter of the coupler ring attached to the donor blood vessel will be different from the vessel opening diameter of the coupler ring attached to the recipient blood vessel. For example, the coupler ring (i.e., third coupler ring) that attaches to the donor blood vessel may have a smaller or larger vessel opening diameter than the coupler ring (i.e., fourth coupler ring) that attaches to the recipient blood vessel. In these cases, the vessel right sizer is used to connect the donor and recipient blood vessels.

In various embodiments, the inner diameter, outer diameter, or both of the tube gradually increases from a first end 2150 of the tube to a second end 2152 of the tube. In a first embodiment, the vessel right sizer includes a tube having a first opening and first thickness at a first end and a second opening and second thickness at a second end, opposite the first end. The outer diameters of the tube at the first and second ends are the same. The inner diameters of the tube at the first and second end are different. The thickness of the tube tapers or becomes progressively thinner as one moves from the second end of the tube towards the first end of the tube.

In a second embodiment, the outer diameters of the tube at the first and second ends are different. The inner diameters of the tube at the first and second ends are the same. The thickness of the tube tapers or becomes progressively thinner as one moves from the second end of the tube towards the first end of the tube.

In a third embodiment, the outer diameters of the tube at the first and second ends are different. The inner diameters of the tube at the first and second ends are different. The thickness of the tube as one moves from the second end of the tube towards the first end of the tube is constant.

The vessel extender (or vessel right sizer) may also be described by its cross-sectional area where, for example, the first vessel opening, second vessel opening, tube or combinations of these do not have circular cross sections. For example, in a fourth embodiment, the vessel right sizer includes a first opening having a first cross-sectional area at a first end and a second opening having a second cross-sectional area at a second end. The second cross-sectional area is greater than the first cross-sectional area.

It should also be appreciated that in various embodiments, the cross-sectional area of the vessel extender (or vessel right sizer) varies between the first and second ends of the tube. For example, as shown in the example of FIG. 21 the cross-sectional area increases as one moves from the first end of the tube towards the second end of the tube.

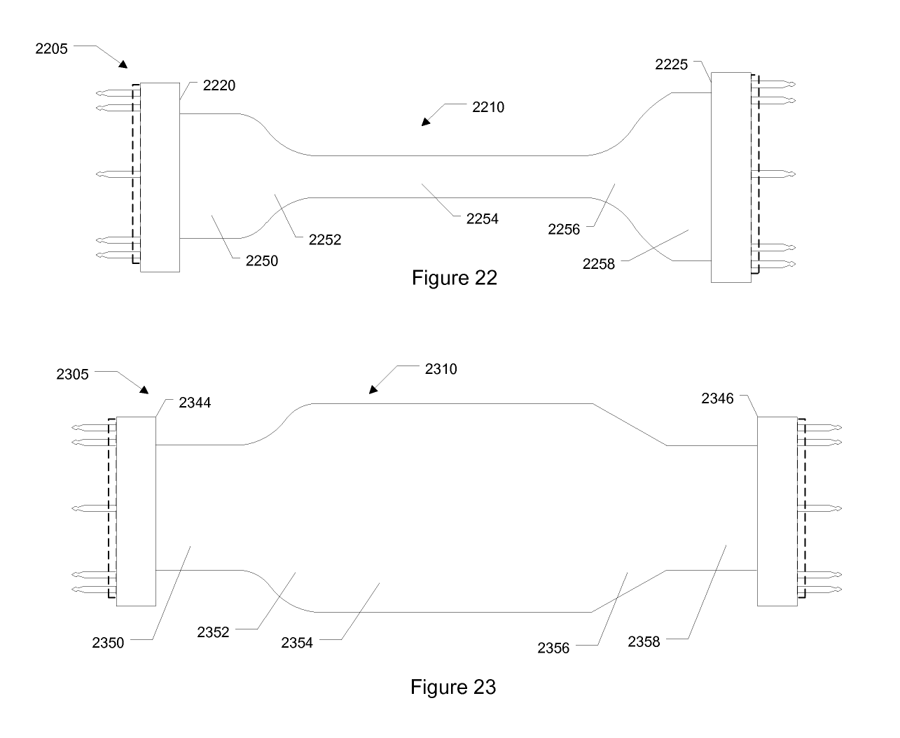

However, this is not always the case. For example, FIG. 22 shows a side view of an implementation of a vessel extender 2205 that includes a tube 2210 that has an hourglass shape. The tube is between coupler rings 2220 and 2225 and includes five portions including a first portion 2250, connected to a second portion 2252, connected to a third portion 2254, connected to a fourth portion 2256, connected to a fifth portion 2258.

A portion may have a cross-sectional area that is different from the cross-sectional area of another portion. Furthermore, a portion may have a constant cross-sectional area while another portion has a cross-sectional area that varies. The cross-sectional area may vary linearly or nonlinearly.

For example, first portion 2250 has a first cross-sectional area that remains constant throughout the first portion. Second portion 2252 has a second cross-sectional area that is less than the first cross-sectional area and decreases at a nonlinear rate. Third portion 2254 has a third cross-sectional area that is less than the second cross-sectional area and remains constant. Fourth portion 2256 has a fourth cross-sectional area that is greater than the third cross-sectional area and increases at a nonlinear rate. Fifth portion 2258 has a fifth cross-sectional area that is less than the fourth cross-sectional area and remains constant.

This specific implementation of the vessel extender may be used to avoid interfering with the position of other structures (i.e., tissue) in the body cavity. For example, third portion 2254 (i.e., the narrow portion of the vessel extender) can be routed between two closely spaced pieces of tissue within the body cavity.

It should be appreciated that various implementations of the vessel extender can include any number of tube portions where each portion has a cross-sectional area that remains constant, increases at linear rate, increases at a nonlinear rate, decreases at a linear rate, or decreases at a nonlinear rate. Furthermore, at least two tube portions may have the same cross-sectional areas. At least two tube portions may have different cross-sectional areas.

FIG. 23 shows a side view of an implementation of a vessel extender 2305 that includes a tube 2310 with a bulge. The tube is between coupler rings 2344 and 2346 and includes five portions including a first portion 2350, connected to a second portion 2352, connected to a third portion 2354, connected to a fourth portion 2356, connected to a fifth portion 2358.

First portion 2350 has a first cross-sectional area that remains constant throughout the first portion. Second portion 2352 has a second cross-sectional area that is greater than the first cross-sectional area and increases at a nonlinear rate. Third portion 2354 has a third cross-sectional area that is greater than the second cross-sectional area and remains constant. Fourth portion 2356 has a fourth cross-sectional area that is less than the third cross-sectional area and decreases at a linear rate. Fifth portion 2358 has a fifth cross-sectional area that is less than the fourth cross-sectional area and remains constant.

Referring now to FIG. 21, the tube includes a surface 2160. As shown in the example of FIG. 21, the surface has a nonlinear slope. At least a portion of the slope is defined by the exponential function y=a^{x}, a>1. The surface may instead or additionally have a linear slope. That is, at least a portion of the slope is defined by the function y=mx+b.

In a specific embodiment, the number of pins on the first coupler ring of the vessel extender is the same as the number of pins on the second coupler ring of the vessel extender. And the number of pin openings on the first coupler ring of the vessel extender is the same as the number of pin openings on the second coupler ring of the vessel extender.

However, this is not always the case. As discussed above, generally the number pins, pin openings, or both on a coupler ring is proportional to the diameter (or cross-sectional area) of the vessel opening of the coupler ring. That is, the greater the diameter of the vessel opening, the greater the number of pins, pin openings, or both. Thus, in implementations where the diameters of the vessel openings for a vessel extender are different, the number of pins on the first coupler ring may be different from the number of pins on the second coupler ring. And the number pin openings on the first coupler ring may be different from the number of pin openings on the second coupler ring.

In a specific embodiment, a vessel extender includes a tube between a first coupler ring and a second coupler ring. The first coupler ring has a first vessel opening with a first diameter, a first number of pins, and a first number of pin openings. The second coupler ring has a second vessel opening with a second diameter, a second number of pins, and a second number of pin openings. The second diameter is greater than the first diameter. The second number of pins is greater than the first number of pins. The second number of pin openings is greater than the first number of pin openings. As an example, the first coupler ring has six pins and six pin openings. And the second coupler ring has eight pins and eight pin openings. As another example, the first coupler ring has four pins and four pin openings. And the second coupler ring has ten pins and ten pin openings.

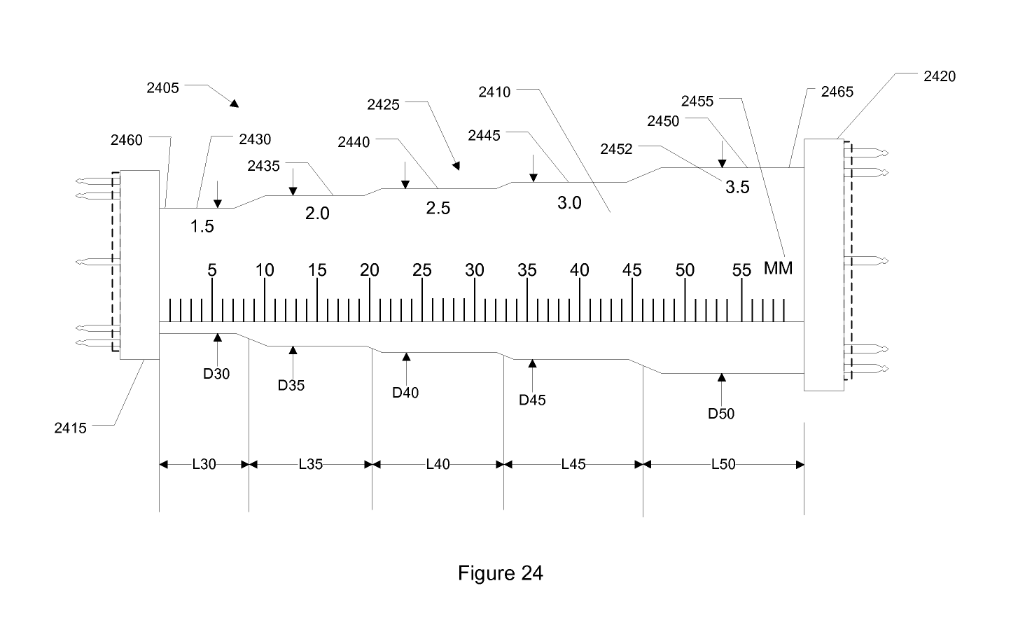

FIG. 24 shows a side view of a vessel right sizer 2405 that includes a tube 2410 connected between coupler rings 2415 and 2420 where the tube includes stepped portions 2425. As shown in the example of FIG. 24, the tube includes five portions including a first portion 2430, connected to a second portion 2435, connected to a third portion 2440, connected to a fourth portion 2445, connected to a fifth portion 2450.

The tube further includes diameter markings 2452 for each of the portions and a set of vertical lines or graduated length markings 2455. The length markings span from a first end 2460 of the tube to a second end 2465 of the tube.

Lengths L30, L35, L40, L45, and L50 indicate the lengths for the first, second, third, fourth, and fifth portions, respectively. In a specific embodiment, lengths L30, L35, L40, L45, and L50 are the same. In another embodiment, at least one length is different from another length.

Diameters D30, D35, D40, D45, and D50 indicate the diameters for the first, second, third, fourth, and fifth portions, respectively. As shown in the example of FIG. 24, these diameters are different and are constant along their respective portions.

In a specific embodiment, the diameter markings include numbers as shown in the example of FIG. 24. In another embodiment, the diameter markings instead or additionally include lines. In this specific embodiment, a first set of lines indicates a length measurement from an end of the tube to a point on the tube and a second set of lines indicates a diameter measurement of the tube portion.

The first and second sets of lines are visually different so that the user can distinguish between the two sets of lines. In various implementations, the first set of lines is in a color different from the second set of lines; the first set of lines at least partially encircle the tube while the second set of lines encircle the tube; the thickness or weight of the first set of lines is different from the thickness or weight of the second set of lines; the first set of lines is in a pattern different from the second set of lines (e.g., solid lines and dotted or dashed lines); or combinations of these.

The second set of lines may include lines with thicknesses or weights that vary proportionally to the diameter of the tube that the line encircles. In a specific embodiment, a first line indicating the diameter of the first portion of the tube encircles the first portion of the tube. A second line indicating the diameter of the second portion of the tube encircles the second portion of the tube. The first and second lines have different thicknesses to indicate the different diameters of the first and second tube portions. The second line is thicker than the first line to indicate that the second portion of the tube has a greater diameter than the first portion of the tube.

Furthermore, the lines in the second set of lines (i.e., the lines indicating tube diameters) may not be equally spaced from each other. For example, in a further embodiment, a third line indicating the diameter of the third portion of the tube encircles the third portion of the tube. The spacing between the first line and the second line is different from the spacing between the second line and the third line.

Generally, the diameters of the portions increase as one moves from the first end of the tube to the second end of the tube. In a specific embodiment, the change in diameter between adjacent portions is constant. For example, as shown in the example in FIG. 24, the change in diameter between adjacent portions (e.g., between the first portion and the second portion, between the second portion and the third portion, between the third portion and the fourth portion, and between the fourth portion and the fifth portion) is an increment amount X where X is 0.5 millimeters. The increment amount can be any number, but ranges from about 0.1 millimeter to about 5 millimeters. Some examples include 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 millimeters, or more than 4.9 millimeters. In a specific embodiment, the increment amount is less than 0.1 millimeter.

A ratio of a smaller diameter (i.e., diameter D30) to a larger diameter (i.e., diameter D35) is typically in the range of about 1:1.1 to about 1:10. In a specific embodiment, the ratio is 1:1.3. Other examples of the ratio include 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.5, 1:6, 1:6.5, 1:7, 1:7.5, 1:8, 1:8.5, 1:9, 1:9.5, or 1:9.9.

In a specific embodiment, the change in diameter among three or more portions is not constant or varies. That is, a first portion has a first diameter, a second portion has a second diameter, and a third portion has a third diameter. A first difference is between the second diameter and the first diameter. A second difference is between the third diameter and the second diameter. The second difference is different from the first difference.

Several benefits of the vessel right sizer is that it can bridge the gap between two body vessels (e.g., donor and recipient blood vessels) that are too short to reach each other and compensate for any difference between the diameters of the two body vessels.

For example, the user may encounter a situation where the donor and recipient blood vessels are too short to reach each other, the diameters of the donor and recipient blood vessels are different, the vessel right sizer is too long, or combinations of these. The vessel opening diameter of the first coupler ring on the vessel right sizer may be desirable, but the vessel opening diameter of the second coupler ring may be too large. Instead, the desired vessel opening diameter may be diameter D40 which is in the third portion. Furthermore, the desired vessel extender length may be somewhere within the third portion as measured from the first end.

The user can cut the vessel right sizer at the third portion and at the desired length along the third portion. This cut end can then be attached to a new coupler ring. The user can then use this new version of the vessel right sizer to connect the donor and recipient blood vessels.

In a specific embodiment, the markings do not correspond to any specific unit of measurement. For example, as discussed above, in a specific embodiment, the markings indicate one or more positions on the tube between the first and second end of the tube. The doctor can use the markings as a reference point for where to cut the tube. For example, the markings may include one or more dotted lines spaced at regular or irregular intervals and which at least partially surround or encircle the tube. The doctor can then use the dotted lines as a reference point for where to cut the tube.

Although FIG. 24 shows five portions, other embodiments include any number of portions such as two, three, four, five, six, seven portions, or more than seven portions.

It should also be appreciated that various implementations include no markings, diameter markings and no length markings, length markings and no diameter markings, or both length and diameter markings.

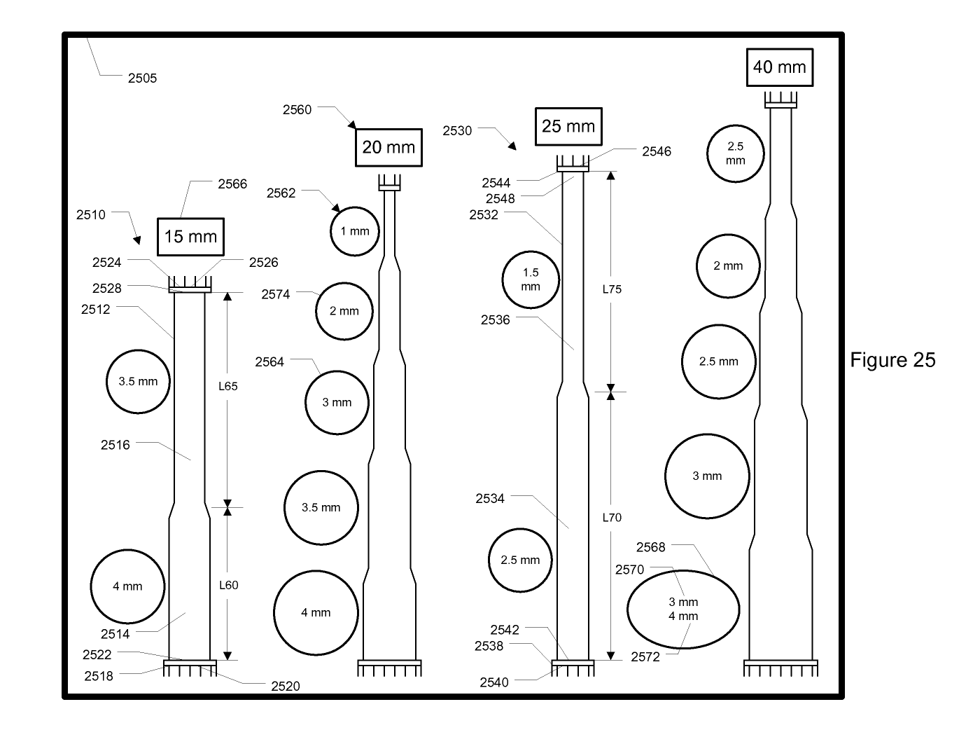

FIG. 25 shows an implementation of a packaging option for the vessel right sizers. A kit 2505 includes an assortment of vessel right sizers with portions having varying lengths and diameters.

In the example shown in FIG. 25, a first vessel right sizer 2510 includes a first tube 2512 that includes a first tube portion 2514 having a first tube portion length L60 connected to a second tube portion 2516 having a second tube portion length L65. A first coupler ring 2518 having a first vessel opening 2520 is connected to a first end 2522 of the first tube portion. A second coupler ring 2524 having a second vessel opening 2526 is connected to a second end 2528 of the second tube portion.

A second vessel right sizer 2530 includes a second tube 2532 that includes a third tube portion 2534 having a third tube portion length L70 connected to a fourth tube portion 2536 having a fourth tube portion length L75. A third coupler ring 2538 having a third vessel opening 2540 is connected to a third end 2542 of the third tube portion. A fourth coupler ring 2544 having a fourth vessel opening 2546 is connected to a fourth end 2548 of the fourth tube portion.

As shown in the example of FIG. 25, the diameters of the first and second vessel openings are different. The diameters of the third and fourth vessel openings are different. The diameter of the first vessel opening is greater than the diameter of the second vessel opening. The diameter of the third vessel opening is greater than the diameter of the fourth vessel opening.

Similarly, the first and second tube portion lengths are different. The third and fourth tube portion lengths are different. The first tube portion length is less than the second tube portion length. The third tube portion length is greater than the fourth tube portion length.

It should be appreciated that many other combinations of tube portion lengths, number of tube portions, and vessel opening diameters are possible. For example, a specific embodiment of the kit includes first and second vessel right sizers. The first vessel right sizer has a first tube with a first portion and a second portion. The first portion has a first length and a first diameter. The second portion has a second length and a second diameter. The second vessel right sizer has a second tube with a third portion and a fourth portion. The third portion has a third length and a third diameter. The fourth portion has a fourth length and a fourth diameter.

In various embodiments, at least two lengths are the same, at least two lengths are different, at least two diameters are the same, or at least two diameters are different.

In a specific embodiment, the kit further includes a first set of labels 2560 having a first shape and a second set of labels 2562 having a second shape, different from the first shape. The difference in shapes indicates the type of dimension being specified. For example, a label 2564 has the shape of a circle to indicate that the dimension being specified is a diameter which is a dimension for a circle.

A label 2566 has the shape of a rectangle to indicate that the dimension being specified is a length (i.e., length of the tube) which is a dimension for a rectangle.

A label 2568 has the shape of an ellipse to indicate that the vessel opening, cross-sectional shape of the tube, or both has the shape of an ellipse or oval. A first number 2570 and a second number 2572 in label 2568 indicate the lengths of the minor and major axis, respectively, of the vessel opening.

In a specific embodiment, the size of the label corresponds to the size of the dimension. That is, the size of the label is proportional to the size of the dimension specified by the label. For example, label 2564 has a greater size (i.e., greater diameter) than a label 2574 because label 2564 specifies a diameter measurement of 3 millimeters which is greater than the diameter measurement of 2 millimeters specified by label 2574.

These visual cues (e.g., shapes and sizes of shapes) provide a user-friendly way to convey information to the user without the user having to read words and numbers and make mental comparisons.

FIG. 26 shows a side view of a sixth embodiment of a vessel extender 2605. Vessel extender 2605 includes a multibranch tube 2610. In a specific embodiment, the multibranch tube is a Y-shaped or bifurcated tube that includes a first tubing branch 2615, a second tubing branch 2620, and a third tubing branch 2625 that are connected together. The ends of the first, second, and third tubing branches are connected to first, second, and third coupler rings 2630, 2635, and 2640, respectively. The first, second, and third coupler rings have first, second, and third vessel openings 2645, 2650, and 2655.

Typically, the angle between second tubing branch 2620 and third tubing branch 2625 is less than 90 degrees. For example, the angle may be about 10, 15, 20, 25, 30, 35, 40, 45, 50, 55, 60, 65, 70, 75, or 80 degrees. In other implementations, the angle may be about 90 degrees or more than 90 degrees.

The diameters or cross-sectional areas of the vessel openings for each of the coupler rings vary depending upon the application. In a specific implementation, the diameters of the first, second, and third vessel openings are the same. In another implementation, the diameter of the first vessel opening is different from the diameter of the second vessel opening and the diameter of the third vessel opening. And, the diameter of the second vessel opening is different from the diameter of the third vessel opening. In another implementation, at least two vessel openings have the same diameter or at least two vessel openings have different diameters.

Likewise, the lengths for each of the tubing branches vary depending upon the application. In a specific implementation, the lengths of the first, second, and third tubing branches are the same. In another implementation, the length of the first tubing branch is different from the length of the second tubing branch and the length of the third tubing branch. And the length of the second tubing branch is different from the length of the third tubing branch. In another implementation, at least two tubing branches have the same length or at least two tubing branches have different lengths.

The length of the multibranch vessel extender may be defined as the longest distance between any two vessel openings. For example, in FIG. 26, the longest distance is between vessel opening 2645 and vessel opening 2655.

In a specific embodiment, vessel opening 2645 is a fluid (e.g., blood) input port and vessel openings 2650 and 2655 are fluid output ports. That is, the vessel extender includes one fluid input port and two fluid output ports. In a specific implementation, the vessel opening that accepts fluid input has a diameter that is greater than the diameters of the two or more vessel openings that produce fluid output. In another implementation, the vessel opening that accepts fluid input has a diameter that is less than the diameters of the two or more vessel openings that produce fluid output.

A flow path 2660 indicates the flow path of a fluid, such as blood, through the vessel extender. A first flow path segment 2662a is from first vessel opening 2645 to a junction or node 2665. A second flow path segment 2662b is from the junction to second vessel opening 2650. A third flow path segment 2662c is from the junction to third vessel opening 2655. A junction is defined as the point at which a flow path is divided into two or more flow paths or the point at which two or more flow paths are combined. In the example of FIG. 26, the first flow path segment indicates flow into the junction. The flow path is then divided into second and third flow path segments which indicate flow out of the junction.

Generally, the mass flow rate into the junction equals the mass flow rate out of the junction. This is represented as:

mᵢₙ=mₒᵤₜ (3)

Applying this principle to the example shown in FIG. 26 gives the following equation:

m₁=m₂+m₃ (4)

where m₁ is the mass flow rate into the junction via first flow path segment 2662a, m₂ is the mass flow rate out of the junction via second flow path segment 2662b, and m₃ is the mass flow rate out of the junction via third flow path segment 2662c.

The mass flow rate is equal to: ρAv, where ρ is the fluid density, A is the cross-sectional area of the tubing branch, and v is the velocity of the fluid through the tubing branch. Inserting these variables into equation (4) gives the equation:

ρ₁A₁v₁=ρ₂A₂v₂+ρ₃A₃v₃ (5)

Since blood is generally incompressible (i.e., the density does not change), ρ₁=ρ₂=ρ₃, and equation (5) is simplified to:

A₁v₁=A₂v₂+A₃v₃ (6)

In a specific embodiment, the vessel extender includes two fluid input ports and one fluid output port. However, it should be appreciated that there may be any number of fluid input and output ports depending, in part, upon the number of tubing branches and the application for which the multibranch vessel extender is intended.

One benefit of vessel extender 2605 is that it can be used to connect bifurcated body vessels. Some cases include connecting two body vessels (e.g., first and second body vessels) to a third body vessel. With the vessel extender, the doctor can connect the first body vessel to the first tubing branch, the second body vessel to the second tubing branch, and the third body vessel to the third tubing branch.

It should also be appreciated that other implementations of the vessel extender include coupling devices that are different from the coupler rings. Various implementations of the vessel extender include compression plate coupling devices, coupling devices with lug closures (e.g., twist to connect), coupling devices that include male connectors, female connectors, or both (e.g., male hose tail connectors and female hose tail connectors), coupling devices that are designed to be crimped, sewn, or glued together, or combinations of these.

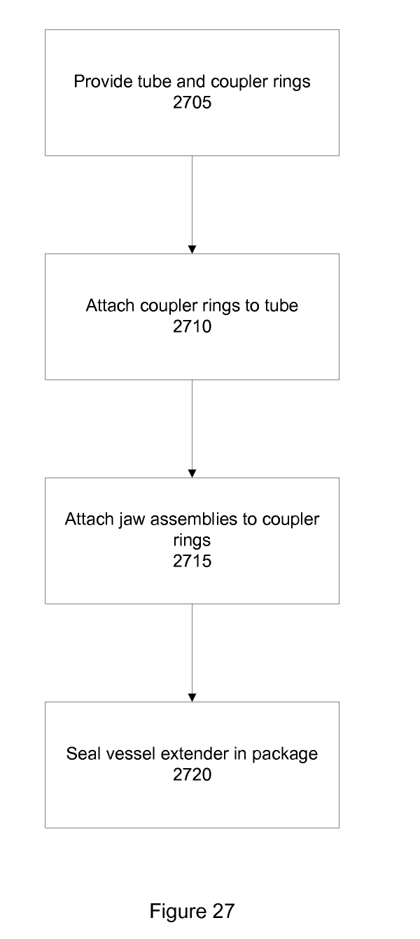

FIG. 27 shows a flow diagram representative of the steps that a manufacturer, doctor, or both may use to create a vessel extender (or vessel right sizer). In a step 2705, first and second coupler rings, and a tube that includes an artificial material, natural material, or both are provided.

The tube is optionally treated with a coating, such as a drug coating. In a specific embodiment, a first portion of the tube is dipped into a container that includes a first drug solution. The thickness of the drug coating is controlled by repeatedly dipping the tube into the container. A second portion of the tube is dipped into a container that includes a second drug solution. This results in a vessel extender that includes two different drug coatings.

In another embodiment, a spray gun is used to apply a drug coating to specific portions of the tube. Masks, for example, are placed along certain portions of the tube during a segment of the coating process to block the drug coating from adhering to a portion of the tube as desired.

In a step 2710, the first and second coupler rings are attached to the tube. For example, a first end of the tube is passed through the vessel opening of the first coupler ring, everted over and impaled onto the pins of the first coupler ring. A second end of the tube is passed through the vessel opening of the second coupler ring, everted over and impaled onto the pins of the second coupler ring. Everting the tube over the pins may include turning the tube outwards and radially stretching the tube towards the pins. That is, typically the tube is made of a material that is stretchable like a balloon.

In a step 2715, a first jaw assembly is attached to the first coupler ring and a second jaw assembly is attached to the second coupler ring.

In a step 2720, the vessel extender including the jaw assemblies is sealed in a package to help maintain a sterile environment inside the package. Labels (e.g., pressure sensitive adhesive labels) which indicate, for example, the size of the vessel extender (e.g., length and diameter) and instructions for handling and storage may be placed on the sterile package.

The user (e.g., doctor) can then open the package at or near the time of use. The vessel extender offers, for example, a prepackaged solution for connecting vessel ends that are too short to reach each other, have varying diameters, or both. The vessel extender can help to save time and cost because, for example, the doctor will not have to perform another surgical procedure (i.e., vessel graft) to obtain a vessel to insert between the two vessel ends. Similarly, the patient will not have to undergo additional surgical procedures. Reducing the number of surgical procedures is generally desirable because it reduces the risk of infection and medical costs.

It should also be appreciated that the vessel extender or vessel right sizer may be made using other manufacturing techniques. For example, the vessel extender (e.g., tube and coupler rings) may be molded (e.g., injection molding) as a one-piece or integrated unit or the tube and coupler rings may be glued together.

In a specific embodiment, the tube is derived using the patient's own cells. A tissue sample is taken from the patient and is used to grow a vessel extender tube. Coupler rings are then attached to the tube. The vessel extender is then preserved and processed for long-term storage. For example, the vessel extender may be cryogenically stored. If at a later time the patient undergoes an operation and the vessel extender is needed, the vessel extender is retrieved from storage and used. Since the tube is derived from the patient's own cells there is generally less of a chance of an allergic reaction or other complication.

Thus, a vessel extender bank much like a sperm bank is maintained for the patient. This can help patients who know that they are predisposed to certain diseases and know that they will likely require surgery at some future date.

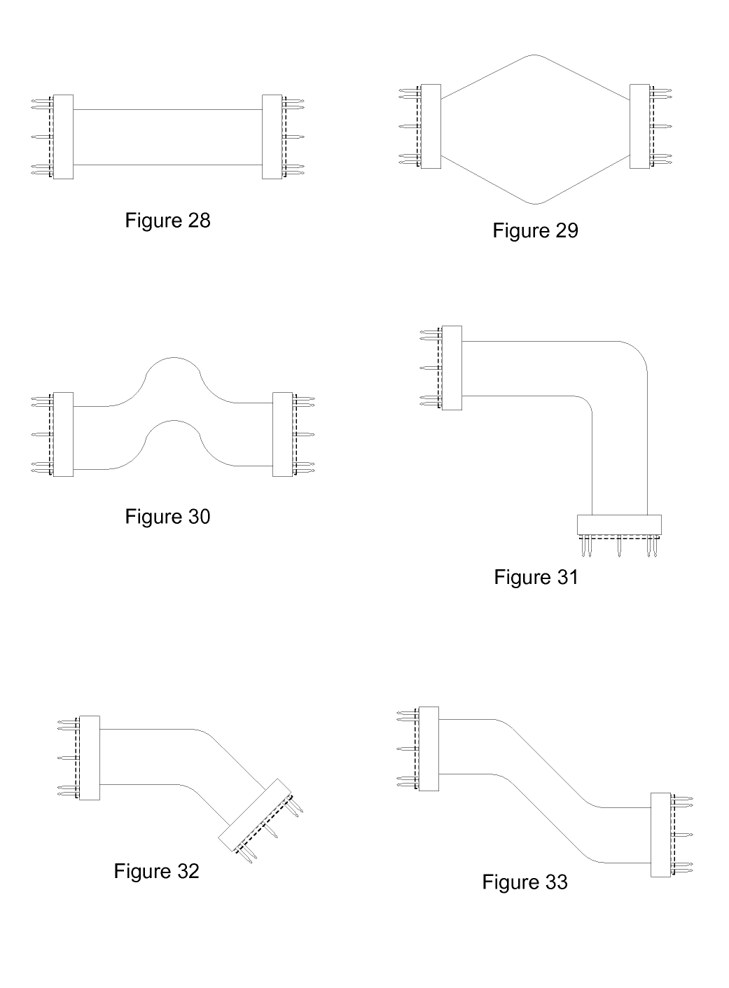

FIGS. 28-38 show sides views of various examples of tube shapes. In a specific implementation, these examples show the tube in a relaxed state. That is, typically, the tube is made of a material that can be elastically, resiliently, or plastically deformed in order to fit within the particular confines of the body cavity. Thus, the tube is generally made of a semirigid or semiflexible material that, in a relaxed or an undeformed state, may have one or more of the shapes as shown in the examples of FIGS. 28-38. However, in another implementation, the tube or portions of the tube are made of rigid materials that may have one or more of the shapes as shown in the examples of FIGS. 28-38.

In a specific implementation, the tube is straight (FIG. 28). However, in various other implementations, the tube has a triangle shape with rounded corners (FIG. 29), a serpentine shape (FIG. 30), a 90-degree elbow (FIG. 31), or a 45-degree elbow (FIG. 32).

FIG. 33 shows a side view of an example of a tube with an offset. That is, a first axis passes through the first coupler ring and is parallel to a first longitudinal axis of a first portion of the tube. A second axis passes through the second coupler ring and is parallel to a second longitudinal axis of a second portion of the tube. The first and second axes are not coincident and are parallel to each other.

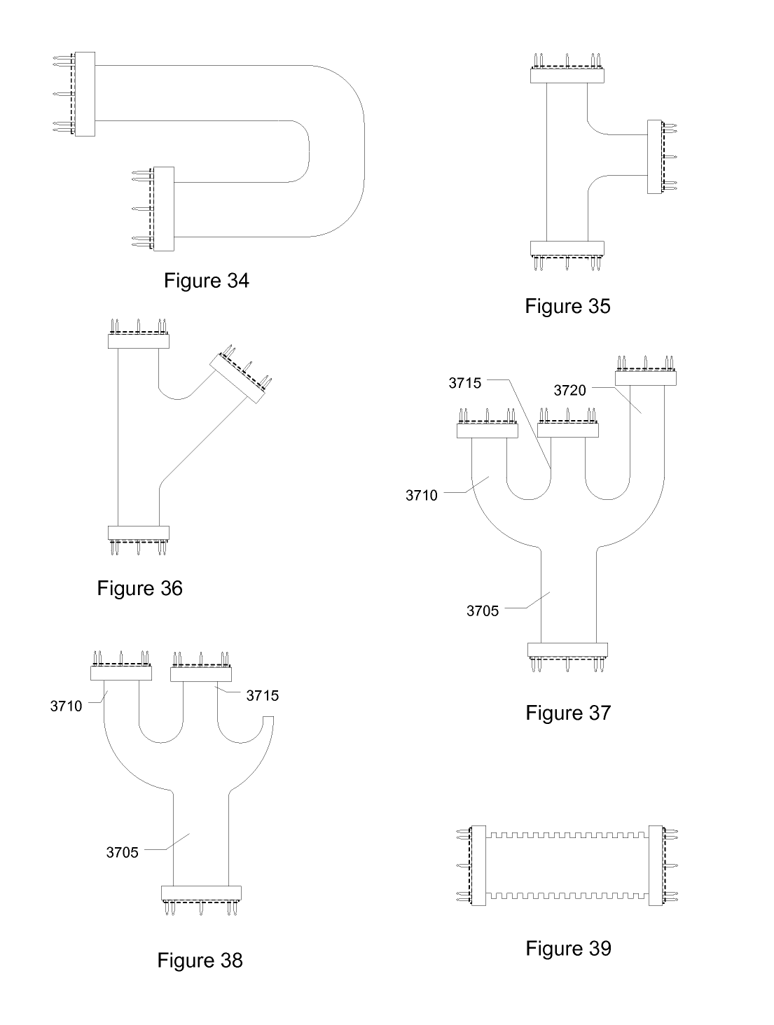

FIG. 34 shows a side view of a tube with a U-shape. FIG. 35 shows a side view of a tube with a T-shape. FIG. 36 shows a side view of a tube with a Y-shape.

FIG. 37 shows a side view of a multibranch tube that includes a trunk portion 3705 coupled to three branch portions 3710, 3715, and 3720.

FIG. 38 shows a side view of the multibranch tube shown in FIG. 37 with branch portion 3720 closed shut (e.g., glued shut, sutured or stitched shut, knotted shut, or tied shut). Such a situation may arise in a case where, for example, the doctor decided that branch portion 3720 was not necessary because of the configuration of the vessels at the surgical site. The doctor may then remove the coupler ring attached to branch portion 3720 and close shut branch portion 3720. Removing the coupler ring includes, for example, cutting, snipping, or clipping branch portion 3720. Removal may also include pulling branch portion 3720 off of the pins of the coupler ring.

FIGS. 39-46 show side views of various examples of corrugated surfaces that a tube may have. FIG. 39 shows a side view of a tube with square shaped corrugations. FIG. 40 shows a side view of a tube with triangle shaped corrugations. FIG. 41 shows a side view of a tube with sawtooth shaped corrugations. FIG. 42 shows a side view of a tube with staircase shaped corrugations.

FIG. 43 shows a side view of a tube having a tube portion that is corrugated and another tube portion that is smooth. FIG. 44 shows a side view of a tube having a corrugated tube portion between smooth tube portions. FIG. 45 shows a side view of a tube having a smooth tube portion between corrugated tube portions. FIG. 46 shows a side view of a tube with tube portions that have different corrugation shapes (e.g., square and triangle shaped corrugations).

The various ideas and concepts presented in this application may be combined, in any combination, with other ideas and concepts presented in this application. For example, the discussion on corrugated surfaces accompanying FIGS. 2-3 is applicable to the implementations of FIG. 21 (vessel right sizer) and FIG. 26 (multibranch tube). The discussion on graduated markings accompanying FIGS. 8 and 24 is applicable to the implementation of FIG. 26 (multibranch tube). The discussion on integrated stents accompanying FIGS. 4-7 is also applicable to the implementations of FIG. 21 (vessel right sizer) and FIG. 26 (multibranch tube). The discussion on examples of tube shapes accompanying FIGS. 28-38 is also applicable to the implementations of FIGS. 2-3 and 39-46 (corrugated surfaces). The discussion on vessel extender kits accompanying FIGS. 17-19 and 25 is also applicable to the implementation of FIG. 26 (multibranch tube).

It should also be appreciated that two or more vessel extenders may be connected together. For example, a first vessel extender may be connected to a second vessel extender. A vessel extender may be connected to a vessel right sizer. A vessel extender with a straight tube may be connected to a vessel extender with a 90-degree elbow. A multibranch vessel extender may be connected to a vessel extender with a tube having a corrugated surface. A first vessel extender without a stent may be connected to a second vessel extender with a stent, and so forth. Thus, the user can create a vessel extender having any desired configuration or feature.

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

The invention claimed is:

1. A device comprising:
a first coupler ring, comprising a first plurality of pins on a first side, a first plurality of pin openings on a second side, opposite of the first side, and a first vessel opening having a first diameter;
a second coupler ring, comprising a second plurality of pins on a third side, a second plurality of pin openings on a fourth side, opposite the third side, and a second vessel opening having a second diameter, wherein the second diameter is the same as the first diameter; and
a tube, coupled between the first and second vessel openings, wherein the tube comprises an artificial material and graduated markings along a portion of the tube.

2. The device of claim 1 wherein the graduated markings comprise a first line at least partially encircling the tube and a second line encircling the tube, wherein the first line indicates a distance from the first vessel opening to the first line and the second line indicates the first diameter.

3. The device of claim 1 wherein the tube comprises:
a first band in a first color; and
a second band in a second color, wherein the second color is different from the first color, the first color indicates a length of the tube, and the second color indicates the first diameter.

4. The device of claim 1 wherein the artificial material comprises at least a polymer based material.

5. The device of claim 1 wherein the artificial material comprises at least a woven material.

6. The device of claim 1 wherein the tube comprises a first coating on a first portion of an inner surface of the tube, wherein the first coating comprises a first drug.

7. The device of claim 6 wherein the tube comprises a second coating on a second portion of the inner surface of the tube, and the second coating comprises a second drug, different from the first drug.

8. The device of claim 1 further comprising:
a first jaw assembly holding the first coupler ring; and
a second jaw assembly holding the second coupler ring.

9. A vessel extender kit comprising a container, comprising a plurality of vessel extenders, wherein a first vessel extender comprises a first coupler ring having a first vessel opening, a second coupler ring having a second vessel opening, and a first tube, coupled between the first and second vessel openings,
a second vessel extender comprises a third coupler ring having a third vessel opening, a fourth coupler ring having a fourth vessel opening, and a second tube, coupled between the third and fourth vessel openings, wherein the first tube has a different length than the second tube.

10. The vessel extender kit of claim 9 wherein the first tube is treated with a cryoprotectant solution.

11. The vessel extender kit of claim 9 wherein diameters of the first and second vessel openings are the same.

12. The vessel extender kit of claim 9 wherein a diameter of the first vessel opening is different from a diameter of the third vessel opening.

13. The vessel extender kit of claim 9 wherein the container comprises a base member, a recloseable lid, and a tray that fits in the base member, the tray having a first cavity to hold the first vessel extender and a second cavity to hold the second vessel extender.

14. The vessel extender kit of claim 13 wherein in the tray, adjacent to the first cavity is a first label and adjacent to the second cavity is a second label, different from the first label.

15. The vessel extender kit of claim 14 wherein the first label indicates a length of the first vessel extender and the second label indicates a length of the second vessel extender.

16. The vessel extender kit of claim 14 wherein the first label indicates a diameter of the first vessel extender and the first label has the shape of a circle.

17. The vessel extender kit of claim 9 further comprising at least one of an irreversible high temperature indicator or an irreversible low temperature indicator attached to a surface of the container.

18. The vessel extender kit of claim 9 wherein the first tube comprises a natural material.

19. The vessel extender kit of claim 18 wherein the natural material comprises a human allograft.

20. The vessel extender kit of claim 18 wherein the natural material comprises a xenograft.

21. The vessel extender kit of claim 18 wherein the natural material comprises a freeze-dried human tissue.

22. A method comprising:
providing a first coupler ring having a first vessel opening and a second coupler ring having a second vessel opening;
providing a tube having a first end and a second end, wherein the tube comprises an artificial material;
attaching the first end of the tube to the first coupler ring;
attaching the second end of the tube to the second coupler ring; and
sealing the tube, first coupler ring, and second coupler ring in a package.

23. The method of claim 22 wherein the package comprises a label attached to an exterior surface of the package, wherein the label comprises information indicating a length of the tube and diameters of the first and second vessel openings.

24. The method of claim 22 wherein before sealing the tube, first coupler ring, and second coupler ring in the package, the method further comprises attaching a first jaw assembly to the first coupler ring, and attaching a second jaw assembly to the second coupler ring.

25. A package to preserve the sterility of contents in the package wherein the contents comprise a vessel extender comprising:
a first coupler ring, comprising a first plurality of pins on a first side, a first plurality of pin openings on a second side, opposite of the first side, and a first vessel opening having a first diameter;
a second coupler ring, comprising a second plurality of pins on a third side, a second plurality of pin openings on a fourth side, opposite the third side, and a second vessel opening having a second diameter, wherein the second diameter is the same as the first diameter; and
a tube, coupled between the first and second vessel openings, wherein the tube comprises a natural material.

26. The device of claim 25 wherein the natural material comprises a human vein or a human artery.

27. The device of claim 25 wherein the natural material comprises a human allograft.

28. The device of claim 25 wherein the natural material comprises a xenograft.

29. The device of claim 25 wherein the natural material comprises a freeze-dried human tissue.

30. The device of claim 25 further comprising:
a first jaw assembly holding the first coupler ring; and
a second jaw assembly holding the second coupler ring.

31. The package of claim 25 further comprising at least one of an irreversible high temperature indicator or an irreversible low temperature indicator attached to a surface of the package.

32. A device comprising:
a first coupler ring;
a second coupler ring;
a tube, comprising a first opening and a second opening, wherein the first coupler ring is coupled to the first opening, the second coupler ring is coupled to the second opening, and the tube is made from a natural material; and
a first marking, visible from an outside of the tube, indicating a length of the tube.

33. The device of claim 32 further comprising a second marking indicating a date of manufacture of the device.

34. The device of claim 32 further comprising a stent within a lumen of the tube.

35. The device of claim 32 further comprising:
a first jaw assembly holding the first coupler ring; and
a second jaw assembly holding the second coupler ring.

36. The device of claim 32 wherein the tube has been treated with a cryoprotectant solution.
